(19) United States
(12) Patent Application Publication
(10) Pub. No.: US 2026/0020925 A1
(43) Pub. Date: Jan. 22, 2026

(54) REMOTE CONTROL OF ROBOTIC-ASSISTED MEDICAL PROCEDURE SYSTEMS USING COMMUNICATION NETWORK CONFIGURATIONS

(71) Applicant: Sovato Health, Inc., Huntington Beach, CA (US)

(72) Inventors: Blair Whitney, Santa Barbara, CA (US); Yulun Wang, Santa Barbara, CA (US); Brian Gaither, South Weber, UT (US); Daniel Wayne Haskell, Santa Barbara, CA (US); Deepam Joshi, Santa Barbara, CA (US)

(21) Appl. No.: 19/191,839

(22) Filed: Apr. 28, 2025

Related U.S. Application Data
(60) Provisional application No. 63/674,187, filed on Jul. 22, 2024.
(60) Provisional application No. 63/741,379, filed on Jan. 2, 2025.

(51) Int. Cl. A61B 34/35; A61B 90/00; G16H 40/67

(52) U.S. Cl. CPC A61B 34/35; A61B 90/361; A61B 90/37; G16H 40/67

(57) ABSTRACT

Disclosed systems, apparatuses, and methods enable remotely controlling robotic-assisted medical systems, such as robotic-assisted surgery systems. Disclosed approaches provide a scalable, flexible, and efficient network for connecting physician consoles and robotic-assisted medical procedure systems. Disclosed approaches improve deployment and safety of robotic-assisted medical procedures.

### CROSS-REFERENCE TO RELATED APPLICATIONS

This applications claims priority to U.S. Provisional Patent Application No. 63/674,187, filed on Jul. 22, 2024, and U.S. Provisional Patent Application No. 63/741,379, filed on Jan. 2, 2025, each of which is incorporated by reference in its entirety.

### TECHNICAL FIELD

This disclosure relates generally to remotely controlling robotic-assisted medical procedures, such as to remotely controlling robotic-assisted surgery systems. Amont others, this disclosure relates to network topology implementation for connecting a physician operating a physician console to a remote robot-assisted medical procedure system.

### DESCRIPTION OF RELATED ART

Robot-assisted surgery systems are generally available and have been developed to operate efficiently and safely. A robotic surgery system typically includes robotically actuable surgical instruments that may be inserted within the patient's body to perform a surgical procedure at a surgical site. The robotic surgery system is typically controlled by a surgeon via a surgeon input console, which is connected to the robotic surgery system via a control cable. The surgeon input console includes input devices that are grasped by the surgeon's hands and moved to generate signals for activating the surgical instruments to perform surgical operations at the surgical site. Signals are transmitted over the control cable to the robotic surgery system, which interprets the signals and generates control signals that cause the instruments to be actuated to perform surgical operations.

### SUMMARY

Disclosed systems, apparatuses, and methods enable remotely controlling robotic-assisted medical procedure systems, such as robotic-assisted surgery systems. Such disclosed approaches provide a scalable, flexible, and efficient network for connecting physician consoles and robotic-assisted medical procedure systems. The network can be tested using one or more simulators. Disclosed approaches improve safety of robotic-assisted medical procedures, such as robotic-assisted surgical procedures.

For physicians or other medical professionals, advantageously, remote procedures facilitate optimal utilization of their time and provide access to a sufficient volume of patients to perfect their skills. For patients, advantageously, remote procedures create ample access to the right physician or medical professional and the right care at an affordable price, decreases the need for travel, and reduces delayed care. Efficient approaches for connecting sites that house robotic-assisted medical procedure systems and sites that house physician consoles that control the robotic-assisted medical procedure systems to a network that enables remote control of robotic-assisted medical procedure systems are disclosed. Disclosed approaches facilitate patient safety and increase the effectiveness of remotely controlled robotic procedures.

Other aspects and features will become apparent to those ordinarily skilled in the art upon review of the following description of specific disclosed implementations in conjunction with the accompanying figures.

### BRIEF DESCRIPTION OF THE DRAWINGS

Specific implementations will now be described with reference to the following drawings, which are provided by way of example.

FIG. 1 is perspective view of a robotic surgery system;

FIG. 2 is perspective view of a robotic surgery system;

FIG. 3 is a block diagram of components of the robotic surgery system of FIG. 2;

FIG. 4 is a schematic view of a remote robotic-assisted medical system;

FIG. 5 is a schematic view of a remote robotic-assisted medical system;

FIGS. 6A and 6B are schematic views of a network for remotely controlling robotic-assisted medical procedures;

FIGS. 6C to 6E are examples of using a network for remotely controlling robotic-assisted medical procedures;

FIG. 7 is a schematic view of a remote robotic-assisted medical system; and

FIG. 8 is a block diagram of a process for planning and establishing medical procedure sessions.

### DETAILED DESCRIPTION

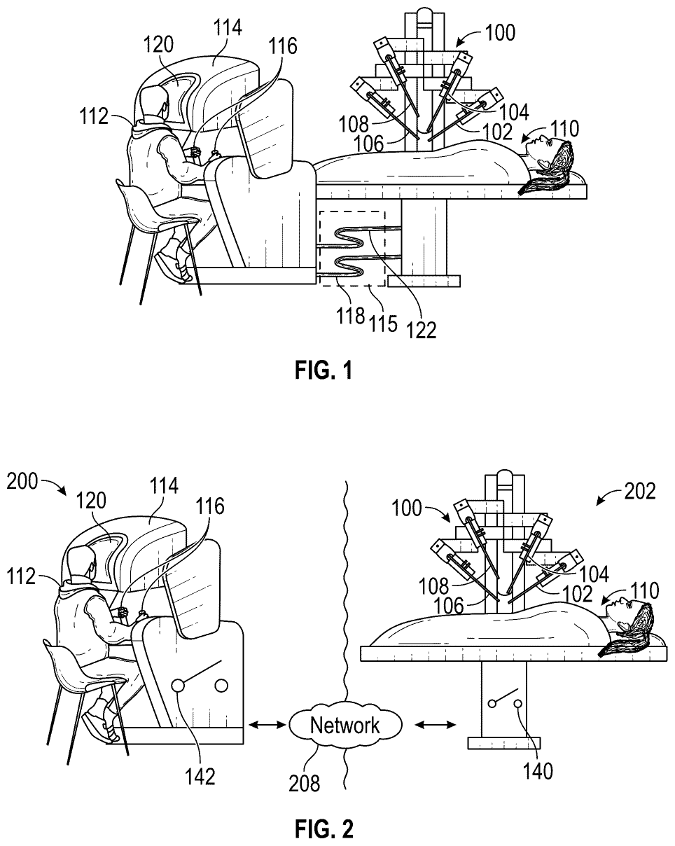

Referring to FIG. 1, a robotic medical procedure system, in particular a robotic surgery system is shown generally at 100. The robotic surgery system 100 includes a plurality of robotically actuable surgical instruments 102-108 positioned on one or more robotic arms. At least one of the instruments 102-108 will generally be configured as an in-patient imaging system (such as, a camera, fluoroscopy system which can obtain a continuous X-ray image, radiography system, computer tomography (CT) system, ultrasound system, magnetic resonance imaging (MRI) system, or the like), which may be inserted into the body of the patient to generate images of anatomical structures and the surgical instruments 102-108 at a surgical site within the body of a patient 110. In some instances, a plurality of imaging systems can be utilized by the robotic surgery system 100. In some cases, one or more imaging systems may not need to be inserted into the patient's body. The remaining instruments may be configured to include an end effector that performs a specific surgical function (such as, forceps/graspers, needle drivers, scissors, electrocautery hooks, staplers, clip appliers, removers, etc.). The system 100 is controlled by a surgeon 112 via a surgeon input console 114, which is connected to the robotic surgery system 100 via a control cable 118. The surgeon input console 114 includes input devices (not shown) including handles 116 that are grasped by the surgeon's left and right hands and moved within an input device workspace or otherwise actuated to generate input signals. The input devices include encoders that transform positions and orientations of the handles 116 into input signal data representing the surgeon input. The input signals may thus be used for activating the plurality of surgical instruments 102-108 to perform surgical operations at the surgical site. The input signals are transmitted over the control cable 118 to the system 100, which interprets the signals and generates control signals that cause the instruments 102-108 to be actuated to move and perform other surgical operations. The input signals will generally be output by the input devices on the surgeon input console 114 as data signals representing the inputs to the console provided by the surgeon 112. For example, the surgeon input console 114 may generate input signals in the form of motion control signals that represent instantaneous positions of the handles 116 of the input devices within an input device workspace. The data signals are transmitted over the control cable 118, which is generally a wired connection between the surgeon input console 114 and the robotic surgery system 100. The wired connection ensures negligible transmission delay such that operations of the surgical instruments 102-108 caused by the surgeon will be effected by the system 100 with negligible delay. Transmission over the control cable 118 may be implemented using any of a variety of different data transmission technologies such as Ethernet or Controller Area Network (CAN bus) protocol.

Additional input signals may also be generated at the surgeon input console 114. For example, the handles 116 may include other controls (not shown) that may be used to generate actuation signals that actuate operations at the surgical instruments 102-108, such as opening or closing a surgical scissor or forceps. The surgeon input console 114 may also include one or more foot pedals (not shown) that may be actuated by the surgeon to initiate various other operations. For example, a foot pedal may be configured to generate a clutch signal for temporarily decoupling the surgical instruments 102-108. A foot pedal may also be configured to initiate delivery of energy, such as generate electrocautery signals for initiating delivery of an electrocauterization current to an instrument, to cut, cauterize, or coagulate tissue (which can involve resection of tissue, vaporization of tissue, or coagulation of tissue). Delivery of energy can include delivery of ultrasonic energy (such as, with a harmonic scalpel instrument), delivery of electric energy (such as, with an electrocautery instrument), delivery of laser energy (such as, with a cautery instrument), delivery of radio frequency energy (such as, with a cautery instrument), or the like. These other input signals also need to be delivered to the robotic surgery system 100 to initiate their respective operations. Additionally, the robotic surgery system 100 may generate event-oriented notifications such as notifications of error conditions that must be communicated to the surgeon input console 114 to update the surgeon 112. Event-oriented messages may be time-sensitive, but are not necessarily synchronous.

The surgeon input console 114 also includes a display 120 for displaying images generated by the in-patient imaging system. The in-patient imaging system would generally be implemented as a high-resolution imaging system (such as, a video camera) that generates a stream of image frames. In some instances, the imaging system may generate images from differing perspectives that convey three-dimensional information and the display 120 may be configured as a stereoscopic display. The display signals generated by the imaging system are transmitted over an image transmission cable 122 back to the surgeon input console 114 for driving the display 120. The image transmission cable 122 is generally selected to ensure that the image frames are delivered to the display in near real-time so that the surgeon 112 does not perceive any delay between their hand movements and movements of the surgical instruments 102-108 represented on the display. In some implementations, the input signals and display signals may both be transmitted over a single shared cable or bus.

A connection 115 can be used for transmission of data between the robotic surgery system 100 and the surgeon input console 114. As illustrated in FIG. 1, the connection 115 can utilize the control cable 118 and the image transmission cable 122. The connection 115 can be a direct connection that links the robotic surgery system 100 and the surgeon input console 114. The connection 115 can be isolated from any other network. For instance, the connection 115 can be a direct local area network (LAN) connection.

The robotic surgery system 100 may be housed within a sterile operating room that forms part of an operating suite. The surgeon or another surgeon aided by a perioperative nurse may make the necessary incisions and insert the instruments 102-108. The surgeon input console 114 may be housed in a portion of the operating suite that is separated from the operating room so that the surgeon operating the input console need not wear surgical gloves while manipulating the controls of the input console. The cables 118 and 122 would generally extend through a port in a wall between the surgeon input console 114 and the robotic surgery system 100. In cases where another surgeon performs the incisions in the body of the patient 110, the operating surgeon may not need to complete the full process of scrubbing, gowning, and gloving before the operation. In this situation, the surgeon input console 114 however remains in a direct wired connection with the robotic surgery system 100 via the cables 118 and 122. The direct wired connection ensures that the robotic surgery system 100 is able to rapidly respond to the surgeon's inputs provided at the surgeon input console 114 and the display 120 displays images of the surgical site with a negligible delay that is virtually unnoticeable to the surgeon.

### Overview of Remotely Controlling Robotic Procedures

While certain examples are described in the context of remotely controlling a surgical procedure, the approaches described herein can be used for remotely controlling a variety of medical procedures, including invasive and non-invasive procedures as well as surgical and non-surgical procedures. The approaches described herein can be used for remotely controlling surgical procedures, interventional procedures, or diagnostic procedures (such as, procedures not involving manipulation of tissue).

One of the main problems in the healthcare industry is the lack of physicians (for instance, surgeons) and their underutilization. On one hand, there is a lack of high-quality physicians. For instance, a 2022 article by the American College of Surgeons concludes that there is an acute ongoing shortage of surgeons available in the United States to serve the patient population. The shortage of high-quality surgical care is particularly severe in rural areas. In addition, surgeons in many geographical areas may not have access to a sufficient volume of patients to perfect their surgical skills because the population is not evenly distributed, thus creating a lack of surgical volume in such areas. On the other hand, currently there are severe inefficiencies with utilizing the time of physicians. For instance, surgeons are required to travel between different hospitals, some of which may be located in difficult to reach places or between different operating rooms in a single hospital. Moreover, surgeons are required to wait for patients and operating rooms to be prepared for surgery. This results in a serious underutilization of surgeons' time. There are many advantages in allowing surgeons to control robotic surgery systems (such as, the system 100) from remote locations in order to increase efficiency and improve patient care. For surgeons, remote surgery facilitates optimal utilization of their time and provides access to a sufficient volume of patients to perfect their skills. For patients, remote surgery creates ample access to the right surgeon and the right care at an affordable price, decreases the need for travel, and decreases delayed care. However, there are a number of challenges with designing a system that would allow remotely controlling robotic surgery systems. These include transmission delay, availability, cumbersome deployment, and reliability of transmission.

Referring to FIG. 2, the surgeon input console 114 is disposed at a surgeon-side location 200 and the robotic surgery system 100 shown in FIG. 1 is disposed at a patient-side location 202. The surgeon-side location 200 is remote from the patient-side location 202 to an extent where a directly wired connection between the robotic surgery system 100 and the surgeon input console 114 is no longer possible. Advantageously, the surgeon-side location 200 may be in another building or even another city, state, or country. In this example, communication is performed between the surgeon-side location 200 and the patient-side location 202 via a network 208, and there is no direct connection 115 between the surgeon input console 114 and the robotic surgery system 100.

The robotic surgery system 100 can include a switching circuitry 140, and the surgeon input console 114 can include switching circuitry 142. The operation of the switching circuitries 140 and 142 is explained below.

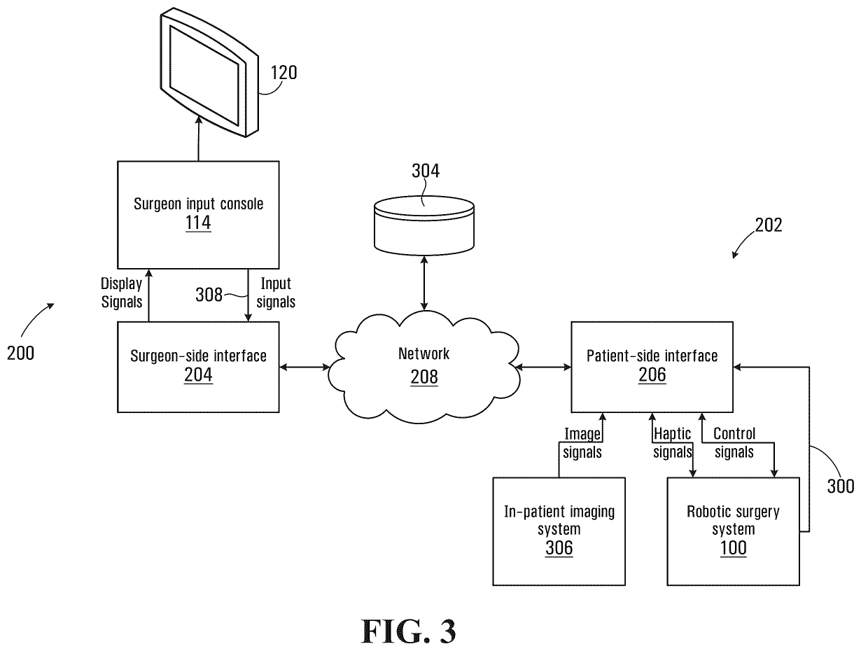

Referring to FIG. 3, communication between the surgeon-side location 200 and patient-side location 202 are conducted via a surgeon-side interface 204 and a patient-side interface 206, which are shown schematically in FIG. 3. The surgeon-side interface 204 is connected to receive input signals from the surgeon input console 114. For example, the surgeon input console 114 may generate a stream of motion input signals 308 that represent the instantaneous position of the handles 116 within an input console workspace. Motion input signals can represent a desired movement (including position and velocity) of an instrument in the input console workspace. These motion input signals will generally be transformed via kinematic processing into signals representing desired positions and orientations of the joints of the instruments 102-108 within a surgical workspace of the robotic surgery system 100. In some instances, kinematic processing translates the position and movement of the handles 116 within the input console workspace into the position and movement of joints of the instruments 102-108. The translation can be performed by transforming the position and orientation of the handles 116 in the Cartesian coordinate system to the coordinates of the joints of the instruments 102-108 in the surgical workspace and vice versa. Other processing functions may then be used to transform these kinematically derived instrument joint positions into drive signals for actuating various actuators, such as motor servos, that cause movement of the instruments 102-108 within the surgical workspace. In the system shown in FIG. 1, the kinematic and other processing may be implemented within the surgeon input console 114 or the within robotic surgery system 100. In the implementation of FIG. 3, the input signals 308 can be picked up directly from the input devices of the surgeon input console 114 and before any kinematic or other processing has been performed. For some robotic surgery system 100, the input signals are generated by the input devices of the surgeon input console 114 at a relatively low frequency (or rate of change), for example about 200 Hz or every 5 milliseconds or about 30 Hz or less. The input signals 308 thus require a relatively low bandwidth or data rate for transmission. In contrast, post-kinematics and other processing signals may have a significantly higher frequency (or rate of change). As an example, in some robotic surgery systems the servo rate at the motor controller may be in the region of about 10 kHz or more, which may require a significantly higher bandwidth or data rate for transmission than the motion input signals generated at the input device.

In general, remotely controlling a robotic surgery system can be achieved by transmitting from the surgeon-side interface 204 to the patient-side interface 206 a complete set of signals that control the instruments 102-108 (which include at least one imaging system). Such signals may be referred to as control signals. To reduce delay and guarantee reliability of the transmission, such set of signals can include signals having the lowest frequency (or rate of change) among a plurality of available signals. As described above, input signals 308 can be transmitted from the surgeon-side interface 204 to the patient-side interface 206, rather than signals generated as a result of kinematic processing that generates signals representing desired coordinates of the joints of the instruments 102-108 in the surgical workspace associated with the robotic surgery system 100 or the drive signals (such as, torque) for actuating the motors of the instruments 102-108. In some cases, the surgeon-side interface 204 can select input signals 308 for transmission from the plurality of available signals, which can include signals generated as a result of kinematic processing and the drive signals.

In certain implementations, one or more signals generated as a result of kinematic processing may be transmitted by the surgeon-side interface 204 to the patient-side interface 206. These signals can be transmitted along with the input signals 308.

The surgeon-side interface 204 is in communication with the patient-side interface 206 over the network 208. The patient-side interface 206 is in communication with the robotic surgery system 100 for receiving and delivering control signals to the robotic surgery system. The patient-side location 202 also includes an in-patient imaging system 306, which generates images of the surgical site within the patient. As described above, one of the surgical instruments 102-108 may be configured to generate the in-patient images or a separate imaging system may be employed. The in-patient imaging system 306 generates data representing image frames, which is encoded into an image data stream by the patient-side interface 206 and transmitted over the network 208 to the surgeon-side interface 204. Image data can be encoded using hardware circuitry of the patient-side interface 204, such as one or more processors, one or more graphics processing units (GPUs), one or more field-programmable gate arrays (FPGAs), or one or more application-specific integrated circuits (ASICs). Image data can include different types of images obtained by different imaging systems. For example, image data can include endoscopic image (or video) data and X-ray images obtained by a fluoroscopy imaging system. Image data can alternatively or additionally include X-ray images obtained by a radiography imaging system, CT images obtained by a CT imaging system, ultrasound images obtained by an ultrasound imaging system, or MRI images obtained by an MRI imaging system. Different types of image data can be encoded into the image data stream for transmission over the network 208 to the surgeon-side interface 204. The surgeon-side interface 204 receives and decodes the image data stream to recover the image frame data, which is sent via the surgeon input console 114 to the display 120 associated with the surgeon input console 114. The surgeon 112 is able to view the surgical site on the display 120 and cause movements of the handles 116 of the surgeon input console 114, which are encoded by the input devices and delivered as control signals to the patient-side interface 206.

The robotic surgery system 100 may be additionally configured to generate haptic feedback and/or force feedback signals. In robotic surgery, haptic feedback signals may be generated to alert the surgeon 112 when an attempt is made to move one of the instruments 102-108 against an instrument movement boundary or other impediment to motion. Haptic signals may also be generated when two of the instruments 102-108 are moved toward a collision condition. These haptic signals are generally used to deliver haptic feedback via the handles 116 of surgeon input console 114 that alert the surgeon 112 to the condition. Additionally, some robotic systems may also be configured to generate force feedback signals that can be used to deliver force feedback to the surgeon via the input console 114. The force feedback may serve to indicate that the surgeon 112 is attempting to move one of the instruments 102-108 against a limitation such as human tissue or an organ. In some implementations the patient-side interface 206 may be configured to receive the haptic or force feedback signals and transmit the signals back to the surgeon-side interface 204 for delivery to the surgeon input console 114.

The network 208 may be provided as a connection to the Internet. In some cases, the network 208 may be a dedicated network such as a point-to-point fiber or a specially-conditioned and monitored network that aims to reduce transmission delay and increase reliability and stability. The network 208 can be a wide-area network (WAN). The surgeon-side interface 204 is in communication with the robotic surgery system 100 at the patient-side location 202 via the network 208.

A data store 304 can be included in communication with the network 208. The data store 304 may be used as remote network storage for storing data logs and other information connected with surgical operations performed by the systems shown.

The robotic surgery system 100 may from time-to-time generate event-oriented notifications at the patient-side location. Each event-oriented notification at the patient-side location 202 may be processed to generate a notification record including a timestamp indicating a time at which the event-oriented notification was generated, status information including information indicating a processing status of the event-oriented notification, and timeout information including information indicating an expiry time by which the event-oriented notification should be processed. These notification records are then transmitted to the surgeon-side location 200. At the surgeon-side location 200, each notification record is received and accumulated for processing. When the status in the notification record indicates that the notification record has not yet been communicated to the surgeon input console and the expiry time in the timeout information exceeds the current time at the surgeon-side location 200 (for instance, as maintained by the surgeon-side interface 204), an alert is generated. When the status indicates that the notification record has not yet been communicated to the surgeon input console and the expiry time does not yet exceed the current time at the surgeon-side location 200, a notification message is generated and communicated to the surgeon input console. The status in the notification record is then changed to indicate that the notification record has been processed.

In some implementations, the robotic surgery system 100 may be expecting the control signals to be provided at a certain threshold rate (such as, the rate of a patient-side synchronization signal (PSS) signal 300). When the control signals received by the robotic surgery system do not satisfy such threshold rate, the patient-side interface 206 can repeat at least some of the received control signals or otherwise modify the control signals in order to provide the control signals to the robotic surgery system 100 at the threshold rate. The threshold rate may not be satisfied as a result of control signals not being received by the patient-side interface 206 at a sufficient rate (because of, for instance, network delay) or due to one or more control signals having been discarded, among others.

In certain cases, the robotic surgery system 100 can transmit acknowledgment information to the surgeon input console 114, for instance, in response to receiving control signals. Acknowledgment information can be transmitted as an acknowledgment signal or message. For example, the robotic surgery system 100 may acknowledge each control signal message or packet with an acknowledgment message. The surgeon input console 114 may be expecting the acknowledgment message to be provided at a certain threshold rate (for instance, corresponding to the rate of transmission of packets to the patient-side location 202). When the acknowledgment messages received by the surgeon input console do not satisfy such threshold rate, the surgeon-side interface 204 can repeat at least some of the received acknowledgment messages or otherwise modify the acknowledgement messages in order to provide the acknowledgment messages to the surgeon-side interface 204 at the threshold rate. The threshold rate may not be satisfied as a result of acknowledgment messages not being received by the surgeon-side interface 204 at a sufficient rate (because of, for instance, network delay) or due to one or more acknowledgment messages having been discarded, among others.

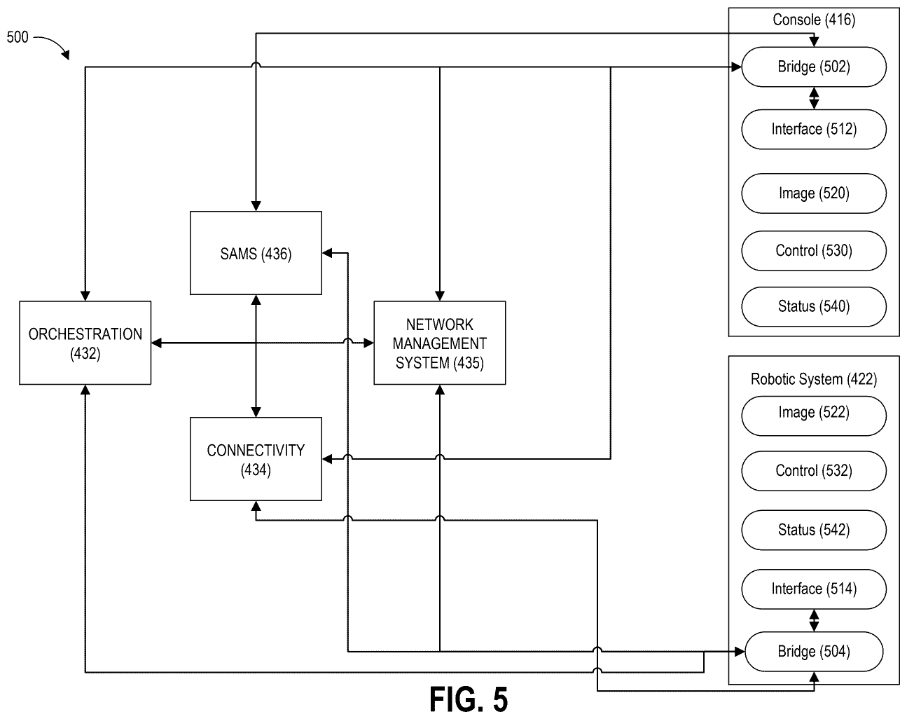

The surgeon-side interface 204 can be integrated with the surgeon input console 114, which can encompass being wholly separate from the surgeon input console 114 or being wholly or partially a portion of the surgeon input console 114. In some implementations, the surgeon-side interface 204 can be implemented as software and/or firmware being executed by one or more processors of the surgeon input console 114. For instance, FIG. 5 illustrates a surgeon input console 416 that implements software and/or firmware interface 512 that provides functionality of the surgeon-side interface 204.

The patient-side interface 206 can be integrated with the robotic surgery system 100, which can encompass being wholly separate from the robotic surgery system 100 or being wholly or partially a portion of the robotic surgery system 100. In some implementations, the patient-side interface 206 can be implemented as software and/or firmware being executed by one or more processors of the robotic surgery system 100. For instance, FIG. 5 illustrates a robotic surgery system 422 (or robot 422) that implements a software and/or firmware interface 514 that provides functionality of the patient-side interface 206.

### System for Remotely Controlling Robotic Procedures

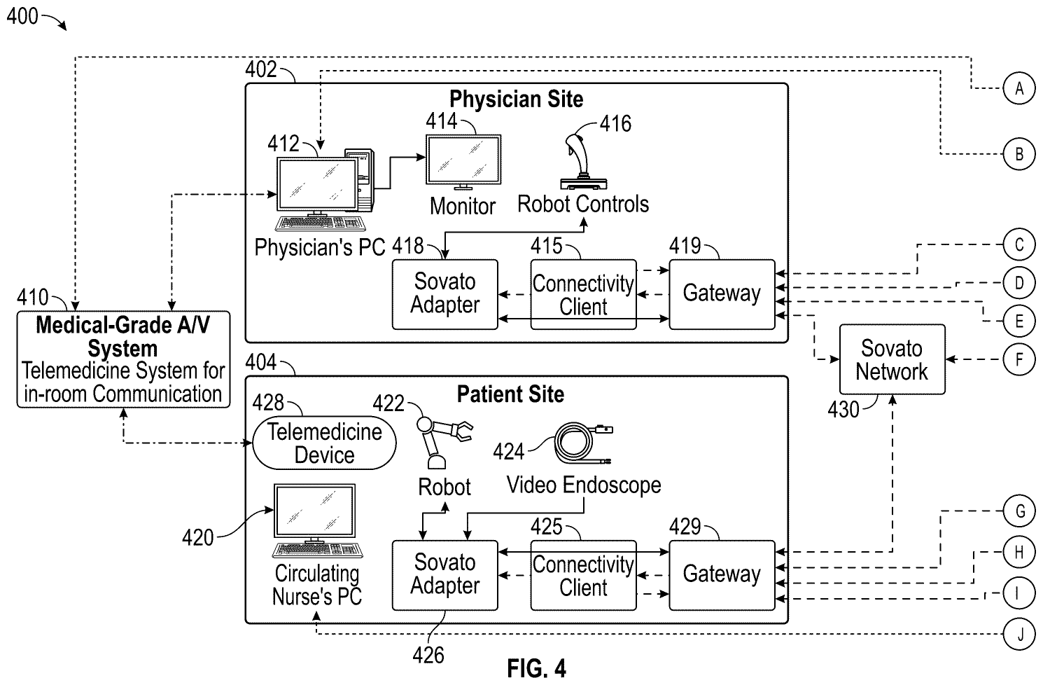

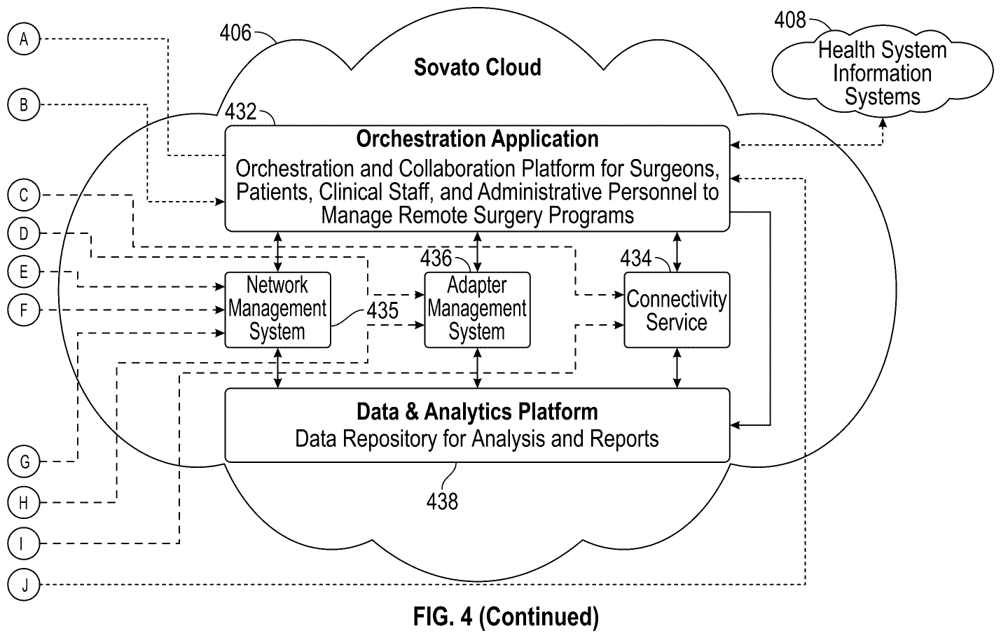

FIG. 4 illustrates a schematic view of a remote robotic-assisted medical system 400. The system 400 can include one or more physician sites 402 each having one or more computing devices 412 (such as, one or more physician personal computers (PCs)), one or more monitors 414 (which can be connected to the one or more physician PCs), one or more physician-side connectivity clients 415, one or more robot control and display systems 416 (which can be, for instance, same as or similar to the surgeon input console 114 and can be referred to as the physician input console 416), one or more physician-side adapters 418 (which can be, for instance, same as or similar to the surgeon-side interface 204), and a physician-side gateway 419. Any of the computing devices described herein can be mobile or portable.

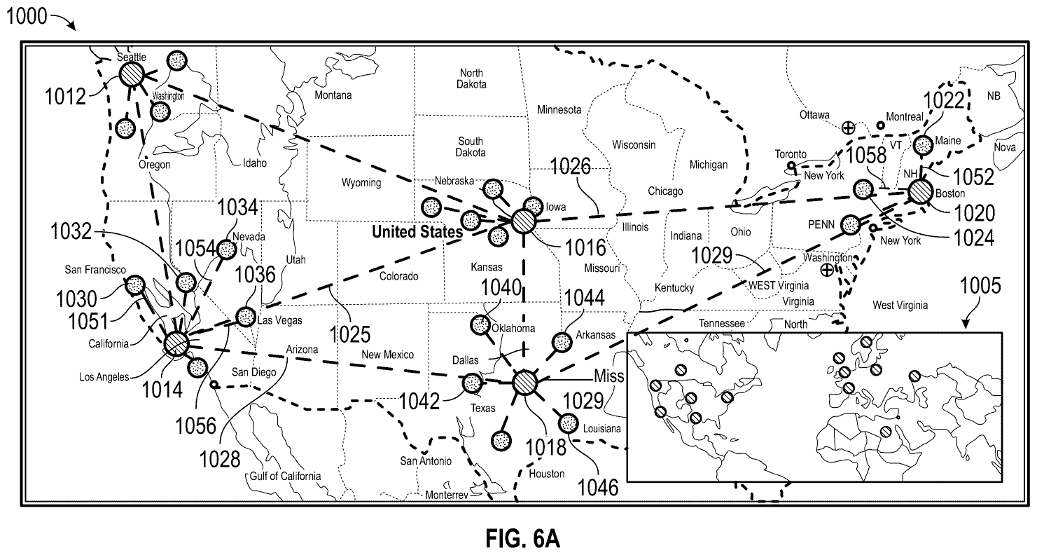

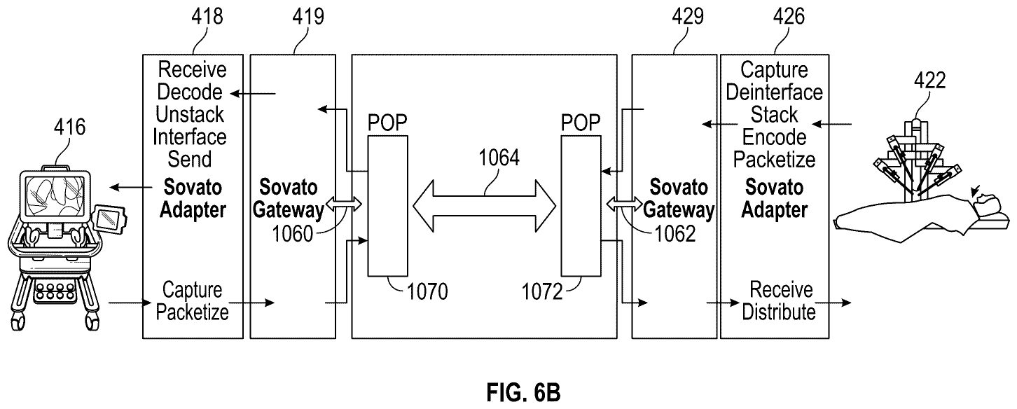

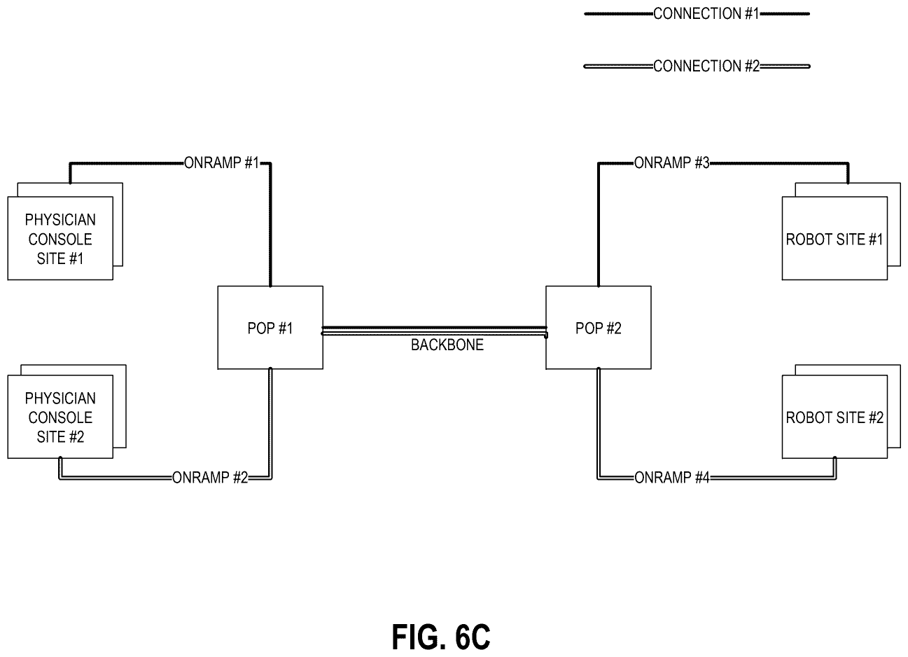

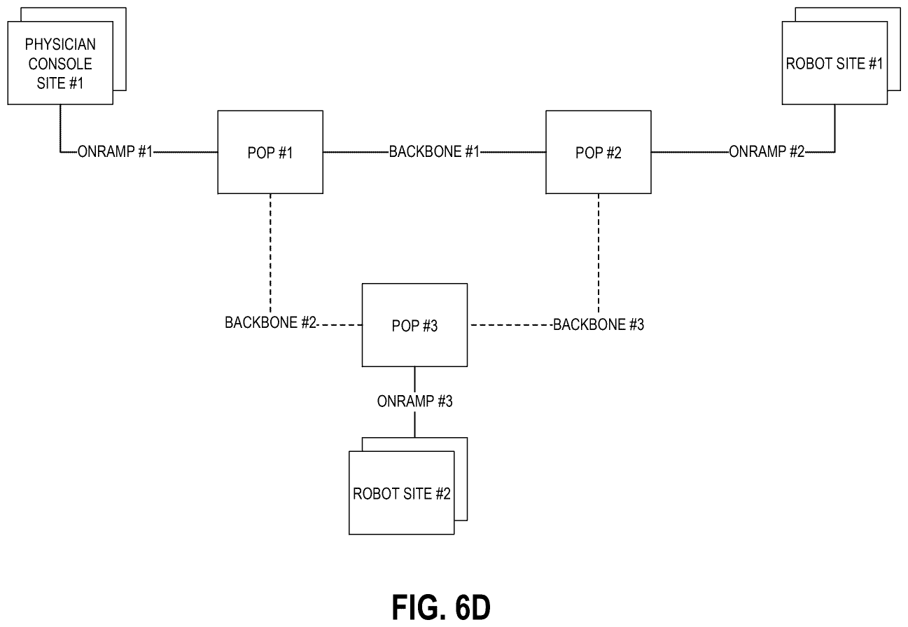

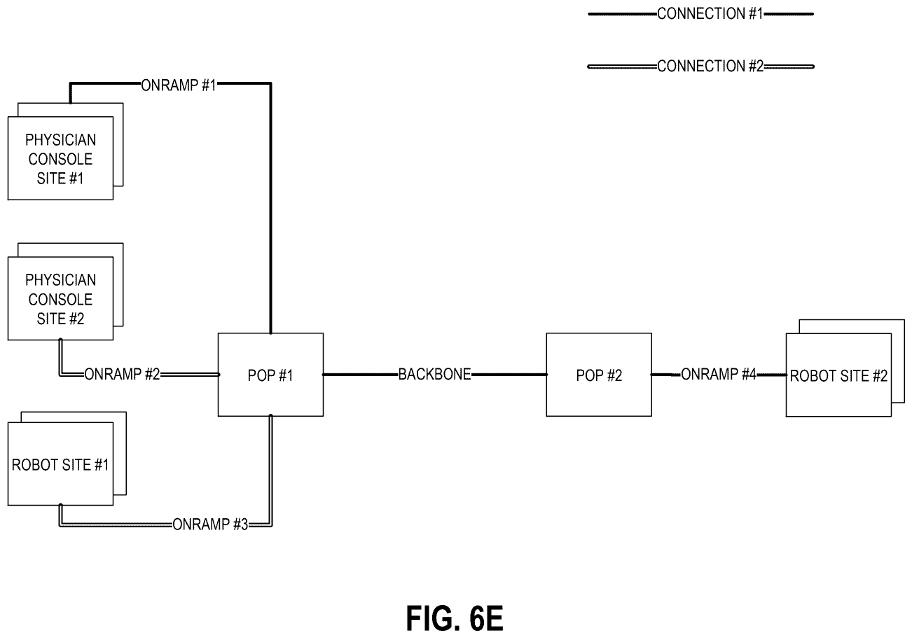

The system 400 can further include one or more patient sites 404 (for instance, medical centers or hospitals) each having one or more computing devices 420 (such as, one or more nurse PCs), one or more medical procedure robots 422 (which can be, for instance, same as or similar to the robotic surgery system 100, and can be referred to as robot), one or more imaging systems 424 (such as, a video endoscope), one or more patient-side connectivity clients 425, one or more patient-side adapters 426 (which can be same as or similar to the patient-side interface 206), and a patient-side gateway 429. The system 400 can also include one or more telemedicine devices 428 configured to communicate telemedicine information and provide the physician with audio and video related to any procedures the physician is performing. The system 400 can further include a network 430 (which can utilize the network 208 as is further explained in connection with FIGS. 6A and 6B) connecting a physician-side adapter 418 to a patient-side adapter 426.

In some cases, as described herein, a physician-side adapter 418 can be integrated with a robot control and display system 416 and/or a patient-side adapter 426 can be integrated with a robot 422. In some instances, a physician PC 412 can be integrated with a robot control and display system and/or a nurse PC 420 can be integrated with a robot 422. Being integrated with can encompass being wholly separate from a corresponding system or being wholly or partially a portion of the corresponding system. For instance, being integrated with can encompass being implemented as software and/or firmware being executed by one or more processors of the corresponding system.

The system 400 can include one or more computing devices, which can form a computing cloud 406. The cloud can implement one or more of: an orchestration server or application 432, a connectivity server 434, a network management system 435, an adapter management server or system 436 (sometimes referred to as SAMS), and a data and analytics server or platform 438. The system 400 can further include a health system information systems 408, comprising a database storing electronic health records (EHR) and/or electronic medical records (EMR) relating to one or more patients. The system 400 can further include one or more medical-grade audio/visual (A/V) systems 410, which can communicate with one or more telemedicine devices 428.

As described herein, the orchestration application 432 can perform one or more of: verify credentials of a physician, provide coordination between the patient side and physician side (or verify that such coordination has been established), establish an A/V connection between the physician side and the patient side (or verify that the A/V connection has been established), verify that a time window for remotely performing a medical procedure on the patient is open, and verify that network connection satisfies one or more conditions. As described herein, the connectivity server 434 can track network addresses of the physician-side adapters 418 and the patient-side adapters 426 and establish a connection between a physician-side adapter and a patient-side adapter. As described herein, the adapter management system 436 can perform adapter monitoring and management. For example, the health of the adapters can be verified and tracked. As described herein, the network management system 435 can facilitate reserving sufficient network resources to guarantee a consistent, predictable transmission of one or more of high-resolution image data, control data, and status data for one or more sessions at once.

A physician PC 412 can be operably connected to a medical-grade A/V system 410 and the orchestration application 432. In an example, the physician PC 412 can communicate telemedicine information and provide the physician with audio and video related to any robotic procedures the physician is conducting. For example, the physician PC 412 can display to the physician on a monitor 414 a real-time video of a patient site 404, the premises where a medical procedure will take place, is being performed, or has been performed. The monitor 414 can display such footage in response to the medical-grade A/V system 410 providing video footage of the patient site 404 to the physician site 402. The physician PC 412 can also be operably connected to the orchestration application 432. The physician PC 412 can transmit physician related information to the orchestration application 432. The physician related information can include information such as the physician's credentials, identification, schedule, specialties, and any access related information related to the robotic procedures the physician will perform. In some implementations, a single physician PC 412 can be used for multiple physician input consoles 416. In some cases, each physician input console 416 can have a dedicated physician PC 412 (for instance, such dedicated physician PC 412 can be connected to the physician input console 416 or its associated physician-side adapter 418 via a wired or wireless connection).

A physician-side connectivity client 415 can allow a physician-side adapter 418 to connect to the network 430 and cloud 406. The physician-side connectivity client 415 can be implemented in software and/or firmware being executed by one or more processors of the physician-side adapter 418. The physician-side connectivity client 415 can call one or more application programming interfaces (APIs) to connect the physician-side adapter 418 to one or more of the network management system 435, the adapter management system 436, or the connectivity server 434. In some instances, the physician-side connectivity client 415 may be implemented by a computing device that is separate from the physician-side adapter 418. In implementations that integrate the physician-side adapter 418 with the physician input console 416, the physician-side connectivity client 415 can be implemented by the physician input console 416.

A physician-side connectivity client 415 (or a physician-side adapter 418) can be assigned an internal internet protocol (IP) address and an external IP address. The internal IP address can correspond to an IP address with respect to a local network of a physician site 402. The external IP address can correspond to an IP address outside of that local network (such as, IP address on the network 430). The physician-side connectivity client 415 can communicate with the cloud 406 via the network 430 and provide information (such as, the internal IP address and the external IP address) to initiate a connection with a patient-side connectivity client 425. The information to initiate a connection with the patient-side connectivity client 425 can include one or more of: an identifier of the physician-side adapter 418, an internal IP address of the physician-side connectivity client 415, or any or information needed to establish a connection with the patient-side connectivity client 425.

A physician input console 416 can include a visual display to show the physician visual indications relating to the robotic procedure. The physician input console 416 can be operably coupled to a physician-side adapter 418. The physician input console 416 can display a view of the robotic procedure site (for instance, as detected by an imaging system 424) and/or any other component relevant to the robotic procedure. A monitor 414 can display visual indicators representing a strength of signal connectivity to the components in the patient site 404. In another example, the monitor 414 can display device characteristics regarding the physician input console 416 and/or the robot 422.

As described in connection with a physician input console 114, a physician input console 416 can include one or more devices used to control one or more medical procedure robots. The physician input console 416 can be operably coupled to a physician-side adapter 418. In an example, robot controls of the physician input console 416 can control movement, actuation, clutch, or any other relevant feature for a medical procedure robot to perform robotic procedures. The physician input console 416, and the corresponding console, can include a type. The type can correspond to the manufacturer of the physician input console, model of the physician input console, and/or version of the software or firmware being executed by one or more processors or the physician input console 416. For example, the manufacturer of the physician input console 416 can include Intuitive Surgical, Medtronic, or any other manufacturer of a physician input console. As another example, the model of the physician input console 416 can include Da Vinci Xi, Da Vinci X, or Da Vinci SP manufactured by Intuitive Surgical.

A physician-side adapter 418 can include a physical device, program, application, application programming interface (API), software development kit (SDK), or another device or program to allow remote robotic procedure. In some implementations, the physician-side adapter 418 can be software and/or firmware being executed by one or more processors of a physician input console 416. The physician-side adapter 418 can be operably coupled to a patient-side adapter 426 via the network 430 and/or the cloud 406, including the connectivity server 434 and the adapter management system 436. In an example, the physician-side adapter 418 can include a device operably coupled to the physician input console 416, to interface the physician input console 416 to the cloud 406 and the network 430. In this example, the physician-side adapter 418 can include hardware components and software components to receive communication signals from the physician input console 416. In this example, the physician-side adapter 418 (or a physician-side connectivity client 415) can include an internal internet protocol (IP) address. The internal IP address can correspond to an IP address with respect to a local network of a physician site 402.

A physician-site adapter 418 and/or a patient-side adapter 426 can be configured software-defined wide-area network (SD-WAN) devices. The network 430 can be configured as SD-WAN network connecting such SD-WAN devices. Internal IP addresses can be used to communicate with devices on the SD-WAN. The internal IP address can be used to establish the connection for remote robotic procedure since the adapters would be part of the same WAN, and external IP addresses may not need to be used. Any other components of the system 400, such as an A/V system 410, physician PC 412, nurse PC 420, or telemedicine device 428, can be similarly configured as SD-WAN devices and become part of the SD-WAN.

Physician-side gateway 419 can forward network traffic between one or more physician-side connectivity clients 415 at a physician site 402 and the network 430. A physician-side connectivity client 415 can be connected to the physician-side gateway 419 via a suitable network connection available at the physician site 402, such as LAN (for instance, implemented using Ethernet or Wi-Fi). In some instances, the physician-side gateway 419 may not be present, and the physician-side connectivity client 415 can be directly connected to the network 430.

A nurse PC 420 can also be operably connected to the orchestration application 432. The nurse PC 420 can transmit patient related information to the orchestration application 432. The patient related information can include the patient's health records, identification, schedule, notes, and any access related information related to the robotic procedures the patient can receive. In some implementations, a single nurse PC 420 can be used for robots 422. In some cases, each robot 422 can have a dedicated nurse PC 420 (for instance, such dedicated nurse PC 420 can be connected to the robot 422 or its associated patient-side adapter 426 via a wired or wireless connection).

A robot 422 can include one or more robotic devices used to perform robotic procedures. The robot 422 can be operably coupled to a patient-side adapter 426. In an example, the robot 422 can receive controls to perform movement, actuation, power, and any other relevant feature to perform a robotic procedure. The robot 422, and any corresponding devices, can include a type. The type can correspond to the manufacturer of the robot, model of the robot, and/or version of the software or firmware being executed by one or more processors of the robot 422. For example, the manufacturer of the robot 422 can include Intuitive Surgical, Medtronic, or any other manufacturer of medical procedure robots. As another example, the model of the robot 422 can include Da Vinci Xi, Da Vinci X, or Da Vinci SP manufactured by Intuitive Surgical.

An imaging system 424 can communicate video to the physician related to any robotic procedures the physician is performing. The imaging system 424 can be operably coupled to the patient-side adapter 426 (directly or through the robot 422). In an example, the imaging system 424 can provide to the physician site 402 a view of a robotic procedure site. The view can include a real-time perspective of the patient before, during, or after robotic procedure is performed.

A patient-side connectivity client 425 can allow a patient-side adapter 426 to connect to the network 430 and cloud 406. The patient-side connectivity client 425 can be implemented in software and/or firmware being executed by one or more processors of the patient-side adapter 426. The patient-side connectivity client 425 can call one or more APIs to connect the patient-side adapter 426 to one or more of the network management system 435, the adapter management system 436, or the connectivity server 434. In some instances, the patient-side connectivity client 425 may be implemented by a computing device that is separate from the patient-side adapter 426. In implementations that integrate the patient-side adapter 426 with the robot 422, the patient-side connectivity client 425 can be implemented by the robot 422.

A patient-side connectivity client 425 (or a patient-side adapter 426) can include an internal IP address and an external IP address. The internal IP address can correspond to an IP address with respect to a local network of a patient site 404. The external IP address can correspond to an IP address outside of that local network (such as, IP address on the network 430). The information to initiate a connection with the physician-side connectivity client 415 can include one or more of: an identifier of the patient-side adapter 426, an internal IP address of the patient-side connectivity client 425, or any other information needed to establish a connection with the physician-side connectivity client 415.

A patient-side adapter 426 can be operably coupled to a physician-side adapter 418 via the network 430 and/or the cloud 406. The patient-side adapter 426 can include one or more of a physical hardware, program, application, API, SDK, or another device or program to allow remote robotic procedures. For example, patient-side adapter 426 can include a device operably coupled to a robot 422 to interface the robot 422 to the cloud 406 and the network 430. In this example, the patient-side adapter 426 can include hardware components and software components to receive communication signals from the robot 422 and establish a network connection to the network 430 and the cloud 406. In some implementations, the patient-side adapter 426 can be software and/or firmware being executed by one or more processors of the robot 422. Similarly to a physician-side adapter 418, the patient-side adapter 426 can include an internal IP address and an external IP address. As described herein, one or more of such IP addresses can be utilized to establish a connection with the physician-side adapter. The information to initiate a connection with the physician-side adapter 418 can include an identifier of the patient-side adapter 426, an internal IP address of the patient-side adapter 426, an external IP address of the physician-side adapter 418, and/or any or information relevant to establish a connection with the patient-side adapter 426. As described herein, the patient-side adapter 426 can be configured as SD-WAN device and its internal IP address can be used to establish the connection.

A telemedicine device 428 can communicate telemedicine information and provide a physician with audio and video related to any robotic procedures the physician is performing. The telemedicine device 428 can be operably coupled to a medical-grade A/V system 410. In an example, the telemedicine device 428 can provide to the physician site 402 a view of the patient site 404 (such as, an operating room). The view can include a real-time perspective of the patient before, during, or after a procedure is performed. In another example, the telemedicine device 428 can receive information from a physician PC 412 through the medical-grade A/V system 410. For example, the telemedicine device 428 can receive information related to the procedure, including one or more of a notice regarding the physician's readiness, status of connectivity, updated patient health records, or scheduling information. In this example, the related information can include the physician's credentials, identification, schedule, specialties, and any access related information related to the robotic procedures the physician will perform. In another example, the telemedicine device 428 can further include an A/V system including a third-party telemedicine hardware provider and medical cart. The A/V system 410 and/or the telemedicine device 428 can facilitate provision of an audio and/or video feed from the patient site 404 to a physician site 402 (and vice versa) as well as facilitate a two-way audio and/or video communication between the patient site 404 and the physician site 402. In some instances, a two-way audio communication between the patient site 404 and the physician site 402 and a one-way video communication from the patient site 404 to the physician site 402 can be utilized.

Patient-side gateway 429 can forward network between one or more patient-side connectivity clients 425 at a patient-site 404 and the network 430. A patient-side connectivity client 425 can be connected to the patient-side gateway 429 via a suitable network connection available at the patient site 404, such as LAN. In some instances, the patient-side gateway 429 may not be present, and the patient-side connectivity client 425 can be directly connected to the network 430.

The network 430 can connect the physician-side adapter 418 and the patient-side adapter 426. The network 430 can be configured as SD-WAN to provide connectivity between the physician-side adapter 418 and the patient-side adapter 426. The network 430 can facilitate a peer-to-peer connection between a physician-side adapter 418 and a patient-side adapter 426. Such peer-to-peer connection can allow the physician-side adapter 418 and the patient-side adapter 426 to transmit and receive information and instructions (such as, in the form of data packets) directly. The information can include robot control instructions and feedback sensor information, real-time patient information including medical health monitoring statuses, patient-side video endoscope content (for example, from the imaging system 424), network connectivity strength including a primary and alternate network connectivity options (for example, SD-WAN, 5G, Ethernet, fiber optic, satellite communication, or any other type of connection available), and any other information relevant to the peer-to-peer network connection.

There can be several ways for establishing a connection via the network 430. For example, a physician-side adapter 418 can request connection to a patient-side adapter 426 from the connectivity server 434. For example, the physician-side adapter 418 can transmit a request to the connectivity server 434 and receive the patient-side adapter 426 internal IP address from the connectivity server 434. In this example, the physician-side adapter 418 can then request to establish a connection (such as, a direct peer-to-peer connection) with the patient-side adapter 426.

As another example, a physician-side adapter 418 can request a connection to a patient-side adapter 426 from the adapter management system 436. For example, the physician-side adapter 418 can transmit a request to the connectivity server 434 and receive the patient-side adapter 426 internal IP address from the adapter management system 436. In this example, the physician-side adapter 418 can then request to establish a connection (such as, a direct peer-to-peer connection) with the patient-side adapter 426. While these examples describe that the physician-side adapter 418 initiates the connection to the patient-side adapter 426, the patient-side adapter 426 can similarly initiate the connection in some implementations.

In any of these examples, external IP address(es) can also be used to establish the connection. As described herein, external IP address(es) can be used when the adapters are not part of the same network (such as, not part of the same SD-WAN).

The connection can be established in response to: 1) verification of login credentials of a physician, 2) completion of coordination between the physician side and patient side (such as, completion of one or more handshake protocols), 3) establishment of a two-way audio communication between the physician side and the patient side, 4) establishment of a video communication from at least the patient side to the physician side, 5) verification of compatibility of physician input console and robot, and 6) verification that the network connection satisfies one or more network conditions (such as, one or more of a network latency threshold, network jitter threshold, network packet loss threshold, and network bandwidth threshold). In some instances, the connection can be established in response to ensuring that at least one of the foregoing conditions, at least two of the forgoing conditions, at least three of the foregoing conditions, at least four of the foregoing conditions, or all of the foregoing conditions have been satisfied. In some cases, the orchestration application 432 can perform the verifications.

A connection establishment protocol can be implemented to form the connection. The connection establishment protocol can be coordinated by the orchestration application 432, for instance, responsive to a request to start a session received from a patient side or physician side. The request can be received through a user interface provided by a physician PC 412 or nurse PC 420. Responsive to the request, conditions for establishing the connection can be verified (as described herein). After it has been verified that the connection can be established, the orchestration application 432 can transmit a request to initiate the connection. The request to initiate the connection can include identifiers of a physician-side adapter 418 and patient-side adapter 426 (for instance, IP addresses of the physician-side adapter 418 and patient-side adapter 426, as described herein). The request to initiate the connection can be transmitted to the connectivity server 434, which can forward a request to begin connection to the physician-side adapter 418. The connectivity server 434 can forward the request to begin connection to the IP address of the physician-side adapter 418. In response to receiving the request to begin connection, the physician-side adapter 418 can generate an offer to connect. The offer can be transmitted to the connectivity server 434, which can forward the offer to the patient-side adapter 426 (for instance, to the IP address of the patient-side adapter 426). The connection can be established responsive to the patient-side adapter 426 receiving the offer.

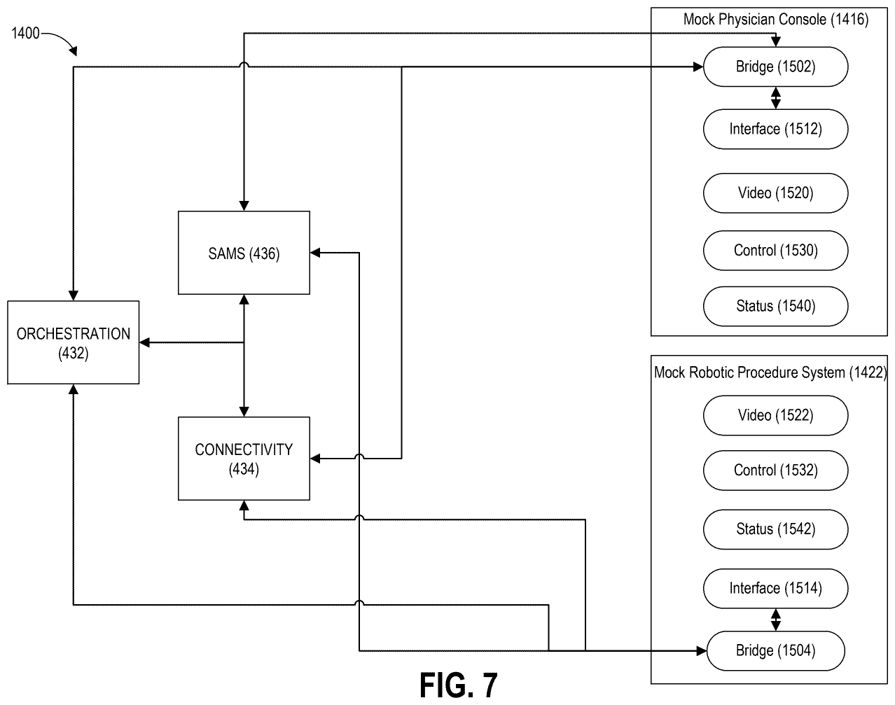

In some instances, the connection establishment protocol can include testing a network connection of one or more the surgeon-side adapter 418 or patient-side adapter 426 using a simulated adapter or a simulated console or robotic surgery system, as described herein in connection with FIG. 7. Such testing can be performed during one or more of initialization of the surgeon-side adapter 418 or patient-side adapter 426 or prior to establishing the connection.

In some implementations, the network 430 can facilitate server-based connectivity (rather than peer-to-peer connectivity). For example, both adapters 418 and 426 can transmit data packets through the connectivity server 434 to communicate with each other.

In some examples, the system 400 can assess whether a type of a robot 422 is compatible with a physician input console 416. In this example, the system 400 will compare the types of the robot 422 and the physician input console 416 to ensure the types are compatible (such as, the same) prior to providing information to establish a connection between the robot 422 and the physician input console 416. In this example, when the robot 422 is of a first type and the physician input console 416 are of the first type, the system 400 will provide over the network 430 information to one or both of the robot 422 and the physician input console 416 to allow the devices to establish a connection (such as, a peer-to-peer connection) for remotely controlling the robot 422. Such connection can be established between the physician-side adapter 418 and the patient-side adapter 426. In another example, when the robot 422 is of a first type and the physician input console 416 are of a second type that is different from the first type, the system 400 will restrict provision of the information over the network 430 and prevent the devices from establishing the connection.

It should be noted that a preliminary maintenance connection may be formed via the network 430 between a physician-side adapter 418 and a patient-side adapter 426 prior to forming the operational connection for remotely controlling the robot 422. The preliminary maintenance connection can be used by the physician-side adapter 418 and the patient-side adapter 426 for exchanging information related to discovery, status, keeping the preliminary connection alive, or the like. This type of connection can be distinct from an operational connection (where the operational connection may be otherwise known as a “session”) for remotely controlling the robot 422 during which the following types of data would be transmitted: control signals (or control commands) transmitted from the physician side to the patient side for moving and/or controlling one or more instruments or imaging systems of a robot 422, status information transmitted from the patient side to the physician side or from the physician side to the patient side (such as, heartbeat signal or other data for ensuring safety), and image data of the procedure site transmitted from the patient side to the physician side. When the operational connection for remotely controlling the robot 422 is established between a physician-side adapter 418 and a patient-side adapter 426, all these three types of data would be exchanged between the physician side and patient side.

Status information can include transmission of signals from the patient side to the physician side and from the physician side to the patient side for monitoring the status of the system 400. For example, the patient side can transmit to the physician side status information indicating that it is operating normally (for instance, this can be a heartbeat signal). As another example, the physician side can transmit to the patient side status information that it is operating normally.

In some instances, a connection for remotely controlling a robot 422 would not be established (and the three types of data described herein would not be exchanged) unless there has been coordination between the patient side and physician side. Coordination can include one or more of performing safety checks (such as, compatibility between the types of the physician input console 416 and the robot 422), ensuring that the patient site (such as, the operating room) and robot 422 have been prepared for a medical procedure, ensuring that the patient has been prepared for the procedure, or the like. In some cases, coordination can be performed by the system 400 and may include completion of or more checklists on the patient side (for instance, by a circulating nurse) and attestation by the physician that the one or more checklists have been completed correctly (such as, by clicking “Attest” on a user interface displayed on the monitor 414). A checklist can be completed on a nurse PC 420, transmitted to the physician side via the network 430, displayed on a physician PC 412, and attested to by the physician through the physician PC 412.

In some instances, at least some types of data related to the connection may be transmitted prior to establishing a connection for remotely controlling a robot 422. For example, image data of the procedure site may be transmitted to help the physician prepare for a procedure (such as, plan the procedure). Transmission of control signals and status information may be commenced at the same time.

The orchestration application 432 can include an orchestration and collaboration platform for physicians, patients, clinical staff, and administrative personnel to manage remote robotic procedure programs. The orchestration application 432 can be executed on one or more remote servers in the cloud 406 and can be operably connected to the health system information system 408, one or more A/V systems 410, one or more physician PCs 412, one or more nurse PCs 420, connectivity server 434, the adapter management system 436, the data and analytics platform 438, and the network management system 435. In some examples, the orchestration application 432 is configured to assess whether a type of a robot 422 is compatible with a physician input console 416.

The assessment of compatibility can occur prior to or at a time of a procedure. For example, the system 400 (such as, via the orchestration application 432) can assess the compatibility at the time the procedure is scheduled, which can be well before the time of procedure (such as, hours, days, or weeks) and well before the connection for remotely controlling the robot 422 is established between the adapters 418 and 426. As a safety check, compatibility may be verified again prior to forming the connection.

In another example, the system 400 can assess compatibility at the time of procedure (such as, close to the initiation of the connection). This can be performed minutes or even seconds prior to the procedure.

The process for the orchestration application 432 to assess the compatibility between a robot 422 and the physician input console 416 can be an automated process, where there is no user intervention to begin the assessment of the compatibility. For example, the orchestration application 432 can pre-verify compatibility of the types prior to when the connection for remotely controlling the robot 422 is established.

The system 400 (such as, via the orchestration application 432) can compare the types of a robot 422 and a physician input console 416 to ensure the types are compatible prior to establishment of the connection for remotely controlling the robot 422. For example, when the robot 422 is of a first type and the physician input console 416 are of the first type, the orchestration application 432 can provide over the network 430 information to one or both of the patient-side adapter 426 and physician-side adapter 418 to allow the devices to establish a connection (such as, a peer-to-peer connection). In another example, when the robot 422 is of a first type and the physician input console 416 are of a second type that is different from the first type, the orchestration application 432 will restrict provision of the information over the network 430 and prevent the devices from establishing the connection.

The connectivity server 434 can include a network signaling server. The connectivity server 434 can be operably coupled to one or more physician-side adapters 418, one or more patient-side adapters 426, the orchestration application 432, and the data and analytics platform 438. The connectivity server 434 can provide a service of connecting the physician-side adapter 418 and the patient-side adapter 426.

For example, the connectivity server 434 can receive a request from a physician-side adapter 418 to establish a connection with a patient-side adapter 426 (or vice versa). In this example, the connectivity server 434 can obtain the physician-side adapter 418 information, including an identifier, an external IP address (if needed), an internal IP address and patient-side adapter 426 information, including an identifier, an external IP address (if needed), and an internal IP address. The connectivity server 434 can obtain the physician-side adapter 418 information from the physician-side adapter 418 and/or from the adapter management system 436. In this example, the connectivity server 434 can also receive an identifier of the patient-side adapter 426 from the physician-side adapter 418, indicating the patient-side adapter 426 with which to establish a connection. The connectivity server 434 can then transmit a request to the patient-side adapter 426, to determine whether the patient-side adapter 426 is capable of establishing a connection to the physician-side adapter 418 over the network 430. In response to receiving an approval from the patient-side adapter 426, the connectivity server 434 can transmit the patient-side adapter 426 information to the physician-side adapter 418 to allow the physician-side adapter 418 to establish a connection between the adapters (for example, via the network 430). In this example, the connectivity server 434 can transmit the patient-side adapter 426 identifier, internal IP address, and external IP address (if needed). The connectivity server 434 can provide a threshold security procedure to assess whether the physician-side adapter 418 has authorization to establish a connection with the patient-side adapter 426. In this example, the connectivity server 434 can include an access control list of authorized adapters and compare the identifier of the physician-side adapter 418 to verify authorization.

In another example, the connectivity server 434 can receive a request from a patient-side adapter 426 to establish a connection with a physician-side adapter 418. The connectivity server 434 can also obtain the patient-side adapter 426 information, including an identifier, an external IP address (if needed), an internal IP address and patient-side adapter 426 information, including an identifier, an external IP address (if needed), and an internal IP address. The connectivity server 434 can obtain the patient-side adapter 426 information from the patient-side adapter 426 and/or from the adapter management system 436. The connectivity server 434 can also receive an identifier of the physician-side adapter 418 from the patient-side adapter 426, indicating the physician-side adapter 418 with which to establish a connection. The connectivity server 434 can then transmit a request to the physician-side adapter 418, to determine whether the physician-side adapter 418 is capable of establishing a connection to the patient-side adapter 426 over the network 430. In response to receiving an approval from the physician-side adapter 418, the connectivity server 434 can then transmit the physician-side adapter 418 information to the patient-side adapter 426 to allow the patient-side adapter 426 to establish a connection between the adapters (for example, via the network 430). In this example, the connectivity server 434 can transmit the physician-side adapter 418 identifier, internal IP address, and external IP address (if needed). The connectivity server 434 can provide a threshold security procedure to assess whether the patient-side adapter 426 has authorization to establish a connection with the physician-side adapter 418. In this example, the connectivity server 434 can include an access control list of authorized adapters and compare the identifier of the patient-side adapter 426 to verify the authorization.

In another example, the connectivity server 434 can receive a request from the orchestration application 432 to establish a connection between a physician-side adapter 418 and a patient-side adapter 426. The connectivity server 434 can obtain the physician-side adapter 418 information, including an identifier, an external IP address (if needed), an internal IP address and patient-side adapter 426 information, including an identifier, an external IP address (if needed), and an internal IP address. The connectivity server 434 can obtain the information from the adapters and/or from the adapter management system 436. For instance, the connectivity server 434 can receive the patient-side adapter 426 identifier from the orchestration application 432 to establish a connection between the patient-side adapter 426 and the physician-side adapter 418. The connectivity server 434 can then transmit a request to the physician-side adapter 418 to determine whether the physician-side adapter 418 is capable of establishing a connection to the patient-side adapter 426 over the network 430. In response to receiving approval from the physician-side adapter 418, the connectivity server 434 can then transmit the physician-side adapter 418 information to the patient-side adapter 426 to allow the physician-side adapter 418 to establish a connection between the adapters (for example, via the network 430). In this example, the connectivity server 434 can transmit the physician-side adapter 418 identifier, internal IP address, and external IP address (if needed) for the patient-side adapter 426 to establish a connection with the physician-side adapter 418. The connectivity server 434 can provide a threshold security procedure to assess whether the adapters have authorization to establish a connection across the network 430. The connectivity server 434 can include an access control list of authorized adapters and compare the identifiers of the adapters to verify authorization.

The adapter management system 436 can provide adapter monitoring and management. The adapter management system 436 can be operably coupled to one or more physician-side adapters 418, one or more patient-side adapters 426, the orchestration application 432, and the data and analytics platform 438. In an example, the adapter management system 436 can store information regarding adapters connected to the cloud 406. The information stored can include identifiers, internal IP addresses, and external IP addresses (if needed) of the adapters. In this example, the adapters connected to the cloud 406 are in a trusted state (for instance, part of the same SD-WAN), such that the adapter management system 436 communicates directly with the adapters. In this example, the adapter management system 436 can communicate with the adapters as if the adapters are on the same local network, such that there is no need to communicate using an external IP address, as described herein.

The data and analytics platform 438 can include a data repository for analysis and reports, which can improve the performance of the medical procedure teams and technology that facilitates remote robotic-assisted procedures. The data and analytics platform 438 can be operably coupled to the orchestration application 432, the connectivity server 434, the network management system 435 and the adapter management system 436. The data and analytics platform 438 can store data from remote robotic procedures performed using the system 400.

The aforementioned components of the system 400 (such as, one or more of the health system information systems 408, a medical-grade A/V system 410, a physician PC 412, a monitor 414, a physician input console 416, a physician-side adapter 418, a nurse PC 420, a robot 422, an imaging system 424, a patient-side adapter 426, the cloud 406, the orchestration application 432, the connectivity server 434, the network management system 435, the adapter management system 436, connectivity clients 415 or 425, gateways 419 or 429, or the data and analytics platform 438) can be communicably coupled to each other via the network 430 and/or the cloud 406, such that data can be transmitted between the components. The network 430 and the cloud 406 can include the Internet, intranet, or other suitable network. The data transmission can be encrypted, unencrypted, over a virtual private network (VPN) tunnel, or other suitable communication means. The network 430 and the cloud 406 can be a wide area network (WAN) (such as, SD-WAN), local area network (LAN), personal area network (PAN), or another suitable network type. The network communication between any of the system 400 components can be encrypted using pretty good privacy (PGP), Blowfish, Twofish, triple data encryption standard (3DES), hypertext transfer protocol secure (HTTPS), or other suitable encryption. The system 400 can be configured to provide communication via the various systems, components, and modules disclosed herein via an application programming interface (API), peripheral component interface (PCI), PCI-Express, American National Standards Institute (ANSI)-X12, Ethernet, Wi-Fi, Bluetooth, or other suitable communication protocol or medium. Additionally, third party systems and databases can be operably coupled to the system components via the network 430 and/or the cloud 406. For example, an EHR/EMR system (such as, the system 408) can be operably coupled to the cloud 406 to transmit patient information, scheduling information relating to a procedure, physician information, or any other relevant information to perform remote robotic procedure.

The data transmitted to and from the components of system 400, can include any format, including JavaScript Object Notation (JSON), transfer control protocol (TCP)/IP, extensible markup language (XML), hypertext markup language (HTML), American Standard Code for Information Interchange (ASCII), short message service (SMS), comma-separated value (CSV), representational state transfer (REST), or other suitable format. The data transmission can include a message, flag, header, header properties, metadata, and/or a body, or be encapsulated and packetized by any suitable format having same.

The network 430 and/or the cloud 406, including the orchestration application 432, connectivity server 434, network management system 435, adapter management system 436, and data and analytics platform 438, can be implemented in hardware, software, or a suitable combination of hardware and software therefor, and may comprise one or more software systems operating on one or more servers having one or more processors with access to memory. The network 430 and/or the cloud 406, including the orchestration application 432, connectivity server 434, network management system 435 adapter management system 436, and data and analytics platform 438, can include electronic storage, one or more processors, and/or other components. The network 430 and/or the cloud 406, including the orchestration application 432, connectivity server 434, network management system 435 adapter management system 436, and data and analytics platform 438, can include communication lines, connections, and/or ports to enable the exchange of information via a network 430 and/or the cloud 406, and/or other computing platforms. The network 430 and/or the cloud 406, including the orchestration application 432, connectivity server 434, network management system 435 adapter management system 436, and data and analytics platform 438, can also include a plurality of hardware, software, and/or firmware components operating together to provide the functionality attributed herein. For example, the network 430 can be implemented by a cloud of computing platforms operating together as the network 430, including Software-as-a-Service (SaaS) and Platform-as-a-Service (PaaS) functionality. Additionally, the cloud 406 can be implemented using commercial cloud computing platforms, including all such functionality provided by the commercial cloud computing platform.

Any of the components of the system 400 can comprise electronic storage that stores information. The electronic storage can include one or both of system storage that can be provided integrally (such as, substantially non-removable) with the network 430 and/or the cloud 406, and/or removable storage that can be removably connectable to the network 430 and/or the cloud 406 via, for example, a port (such as, a Universal Serial Bus (USB) port, a firewire port, etc.) or a drive (such as, a disk drive, etc.). Electronic storage may include one or more of optically readable storage media (such as, optical disks, etc.), magnetically readable storage media (such as, magnetic tape, magnetic hard drive, floppy drive, etc.), electrical charge-based storage media (such as, erasable electronic programmable read only memory (EEPROM), random access memory (RAM), etc.), solid-state storage media (such as, flash drive, etc.), and/or other electronically readable storage media. Electronic storage may include one or more virtual storage resources (such as, cloud storage, a virtual private network, and/or other virtual storage resources). The electronic storage can include a database, or public or private distributed ledger (such as, blockchain). Electronic storage can store machine-readable instructions, software algorithms, control logic, data generated by processor(s), data received from server(s), data received from computing platform(s), and/or other data that can enable server(s) to function as described herein. The electronic storage can also include third-party databases accessible via the network 430 and/or the cloud 406.

Any of the components of the system 400 can include control circuitry, such as processor(s) or controller(s), configured to provide data processing capabilities, for instance, in the network 430 and/or the cloud 406. As such, any of the processors can include one or more of a digital processor, an analog processor, a digital circuit designed to process information, an analog circuit designed to process information, a state machine, and/or other mechanisms for electronically processing information, such as field programmable gate arrays (FPGAs) or application specific integrated circuits (ASICs). The processor(s) can be a single entity or include a plurality of processing units. These processing units can be physically located within the same device, or processor(s) can represent processing functionality of a plurality of devices or software functionality operating alone, or in concert. A networked computer processor can be a processor operably coupled to the network 430 and/or the cloud 406. The networked computer processor can be operably coupled to other processors, databases, or components.

The orchestration application 432, connectivity server 434, network management system 435 adapter management system 436, and data and analytics platform 438 can be configured with machine-readable instructions having one or more functional modules. The machine-readable instructions can be implemented on one or more servers, having one or more processors, with access to memory. The machine-readable instructions can be a single networked node, or a machine cluster, which can include a distributed architecture of a plurality of networked nodes. The machine-readable instructions can include control logic for implementing various functionality, as described in more detail below. The machine-readable instructions can include certain functionality associated with the system 400. Additionally, the machine-readable instructions can include a smart contract or multi-signature contract that can process, read, and write data to the database, distributed ledger, or blockchain.

FIG. 5 illustrates a schematic view of a remote robotic-assisted medical system 500, which can be similar to the system 400. As is described herein, in system 500, the functionality of a physician-side adapter 418 can be implemented in software and/or firmware by an interface 512, and the functionality of a patient-side adapter 426 can be implemented in software and/or firmware by an interface 514. The interface 512 can be executed by one or more processors of the physician console 416, and the interface 514 can be executed by one or more processors of the robot 422. In some instances, the interfaces 512 and 514 can be SDKs. The functionality of a physician-side connectivity client 415 can be similarly implemented by the interface 512, and the functionality of a patient-side connectivity client 425 can be similarly implemented by the interface 514.

A physician console 416 can include a bridge 502 (which can be implemented in software and/or firmware) that connects the interface 512 with one or more of the orchestration application 432, connectivity server 434, network management system 435, or adapter management system 436. As described herein, the physician console can receive image data 520 from the robot 422, provide control signals 530 to the robot, and exchange status information 540 with the robot. In some instances, the bridge 502 can function as or similarly to the gateway 419, which can forward traffic between the interface 512 and one or more of the orchestration application 432, connectivity server 434, network management system 435 or adapter management system 436.

A robot 422 can include a bridge 504 (which can be implemented in software and/or firmware) that connects the interface 514 with one or more of the orchestration application 432, connectivity server 434, network management system 435 or adapter management system 436. As described herein, the robot can provide image data 522 to the physician console 416, receive control signals 532 from the physician console, and exchange status information 542 with the physician console. In some instances, the bridge 504 can function as or similarly to the gateway 429, which can forward traffic between the interface 514 and one or more of the orchestration application 432, connectivity server 434, network management system 435 or adapter management system 436.

### Network for Remotely Controlling Robotic Procedures

Interconnecting physician consoles and robotic procedure systems can involve considerable expense and effort. For instance, suppose that a patient site (such as, a hospital or another suitable site) that houses one or more robotic procedure systems (such as, robotic surgery systems) desires to allow such one or more robotic procedure systems to be controlled by one or more remotely located physician consoles (such as, surgical consoles). To reduce transmission delay and guarantee reliability and stability of transmission, a high-quality network connection is required rather than using the public Internet, and therefore the patient site would need to design and deploy a suitable network (such as, the network 430) that provides one or more connections between the one or more robotic procedure systems and consoles. A physician site (such as, surgeon center or another suitable site) housing physician consoles may need to undergo a similar process. Undesirably, such undertaking can be difficult, time consuming, and expensive. Further, each patient site and physician site that wishes to provide remotely controlled robotic medical procedures may need to design and deploy its own network, which can lead to wasteful duplication of effort and resources.

To address these shortcomings, a network for remotely controlling robotic procedures can be designed and deployed. Such network can include one or more nodes for connecting patient sites and physician sites. A node can be referred to as a point-of-presence (PoP) node (which can also be referred to as a data center). To connect to the network, patient sites and physician site may only need to employ a suitable last mile (or last kilometer) connection that satisfies network requirements for remote robotic procedures (which are described herein). For example, a patient site or physician site center may utilize a dedicated connection (such as, a leased line) for connecting with a hub of the network. The network can be a single network covering a particular geographical area or region (for instance, the continental United States or other regions). Advantageously, this approach can simplify and streamline deployment of remotely-controlled robotic procedure systems.

FIG. 6A illustrates a network 1000 for remotely controlling robotic procedures. The network 1000 is illustrated as covering the continental United States. Another network 1005 is illustrated as covering the continental United States, Canada, Europe, Asia, and Africa. The network 1000 can be a subset of the network 1005. The network 1000 can include a plurality of PoP nodes 1012, 1014, 1016, 1018, and 1020. The PoP nodes can serve as regional hubs for connecting patient sites (such as, hospitals) and physician sites (such as, surgeon centers). For instance, a plurality of sites 1030 and 1032 that house robotic procedure systems (or robotic surgery systems, which can be similar to the robotic surgery system 422) and a plurality of sites 1034 and 1036 that house physician consoles (or surgeon consoles, which can be similar to the surgeon consoles 416) can utilize the PoP node 1014 for connecting to the network 1000. At the same time, a plurality of sites 1040 and 1042 that house physician consoles and a plurality of sites 1044 and 1046 that house robotic procedure systems (1044 and 1046) can utilize the PoP node 1018 for connecting to the network 1000. As is described herein, the sites 1030, 1032, 1034, and 1036 can each be connected to the PoP node 1014 via a suitable last mile connection, and the sites 1040, 1042, 1044, and 1046 can each be connected to the PoP node 1018 via a suitable last mile connection (which can be referred to as onramp connections or onramps).

The network 1000 can guarantee that a physician console located anywhere in an area, country, or region covered by the network can connect (provided that all other conditions described herein are satisfied, for instance, compatibility) to a robotic procedure system located in another part of the area, country, or region covered by the network. To achieve this, a PoP node of the network 1000 can be connected to at least one other PoP node. Some PoP nodes can be connected to multiple other PoP nodes, which can provide failover and redundancy. PoP nodes can be connected to one another by a backbone connection, such as, 1024, 1026, 1028, or 1029. The backbone connection can be at least one of a high-speed direct connection or a high-speed dedicated connection, as described herein.

For example, suppose that a first physician operating a first physician console located at the site 1034 wishes to perform a remote medical procedure on a first patient using a first robotic system located at the site 1022. A first session would be established between the first physician console and the first robotic system by traversing an onramp 1054 connecting the site 1034 and the PoP node 1014, the backbone connection 1024 connecting the PoP node 1014 to the PoP node 1016, the backbone connection 1026 connecting the PoP node 1016 to the PoP node 1020, and an onramp 1052 connecting the site 1022 to the PoP node 1020.

Continuing with the previous example, suppose that a second physician operating a second physician console located at the site 1036 wishes to perform a remote medical procedure on a second patient using a second robotic system located at the site 1024. A second session would be established between the second physician console and the second robotic system by traversing an onramp 1056 connecting the site 1036 and the PoP node 1014, the backbone connection 1024 connecting the PoP node 1014 to the PoP node 1016, the backbone connection 1026 connecting the PoP node 1016 to the PoP node 1020, and an onramp 1058 connecting the site 1024 to the PoP node 1020.

As described herein, the first and second sessions can be concurrent (or simultaneous) and independent of one another. Transmission of control or image data for the first session is separate from transmission of control or image data for the second session. That is, even though data for the first and second sessions is transmitted over shared physical network connections, the data remains distinct and is correctly directed to its intended recipient. Simultaneous sessions are not limited to the above example and can be established between any number of various pairs of compatible physician consoles and robotic systems.

For example, with reference to FIG. 6C, suppose that a physician operating a physician console located at the site labeled “physician console site #1” wishes to remotely control a robotic system located at a site labeled “robot site #1.” To facilitate this, a first connection (illustrated as “connection #1”) traversing an onramp connection 1 connecting the physician console site 1 to PoP 1, a backbone connection connecting PoP 1 and PoP 2, and an onramp connection 3 connecting the robot site 1 to PoP 2 would be established. Suppose further that a physician operating a physician console located at site labeled “physician console site #2,” which is connected to PoP 1 via an onramp connection 2, wishes to remotely control a robotic system located at a site labeled “robot site #2,” which is connected to PoP 2 via an onramp connection 4. To facilitate this, a second connection (illustrated as “connection #2”) traversing the onramp connection 2, the backbone connection connecting PoP 1 and PoP 2, and the onramp connection 4 would be established. The second connection can be established and maintained simultaneously with and independent of the first connection. For example, the second connection can be established before, during, or after the first connection and terminated before, during, or after the first connection.

With reference to FIG. 6A, in some cases, one or more of the first or second sessions is established by traversing the PoP node 1018 instead of the PoP node 1016. In such case, the backbone connection 1028 connecting the PoP node 1014 to the PoP node 1018 and the backbone connection 1029 connecting the PoP node 1018 to the PoP node 1020 would be traversed. This path can be used for redundancy and failover. For instance, responsive to determining that the PoP node 1016 is underperforming or one or more of its associated connections are underperforming, one or more of the first or second sessions can be rerouted through the PoP node 1018. Determination of underperformance can be made by comparing one or more performance metrics (such as, bandwidth, latency, packet loss, or low jitter) to one or more thresholds. Underperformance can be due to oversubscription or one or more technical issues.

Another example of redundancy and failover is illustrated in FIG. 6D that shows that a first connection between a physician console located at a site labeled “physician console site #1” and a robotic system located at a site labeled “robot site #1” can be established in one of two ways: 1) via the associated onramp connections 1 and 2 and a backbone connection 1 connecting PoP 1 to PoP 2 or 2) via the associated onramp connections 1 and 2, a backbone connection 2 connecting PoP 1 and PoP 3, and a backbone connection 3 connecting PoP 3 and POP 2. While the second option is longer because it traverses an additional PoP node (PoP 3), it may be preferable in some cases to the first option.

Same or different physician console located at physician console site 1 may be connected via a second connection to a robotic system located at a site labeled “robot site #2.” The second connection can span the onramp connection 1, backbone connection 2, and onramp connection 3 connecting the robotic system to PoP 3. If the second session is with a different physician console as the first connection, it can be established and maintained simultaneously with and independent of the first connection. If the second connection is with the same physician console as the first connection, first and second connection may not be simultaneous.

With reference to FIG. 6A, the site 1034 or 1036 can house additional physician consoles that may be connected to additional robotic systems located at the sites 1022 or 1024.

A PoP node may be implemented as one or more servers, such as (such as, one or more servers housed in a data center) or can be implemented as software and/or firmware being executed by one or more processors, such as one or more processors of server(s). A data center can house one or more firewalls and one or more network switches (such as, fiberoptic network switches).

FIG. 6B illustrates a remote robotic procedure session that utilizes the network 1000. A physician input console 416 is illustrated as remotely controlling a robot 422. A physician-side adapter 418 is illustrated as being connected to a PoP node 1070 via a gateway 419, which can be optional. A patient-side adapter 426 is illustrated as being connected to a PoP node 1072 via a gateway 429, which can be optional. The gateways 419 and 429 can forward network traffic to the respective PoP nodes 1070 and 1072. In some instances, the gateways 419 and 429 can forward traffic to/from different networks utilized by last mile connections (or onramps) 1060 and 1062 terminating at the gateways 419 and 429 (or the adapters 418 and 426 when the gateways are not present) from/to a network connecting the PoP nodes 1070 and 1072. The gateway 419 can positioned in a physician site along with the physician-side adapter 418 and can be connected to the PoP node 1070 by the onramp connection 1060. The physician-side adapter 418 can be connected to the gateway 419 via a suitable network connection available at the physician site, such as LAN (which can be implemented, for instance, using Ethernet or WiFi). The gateway 429 can be positioned in a patient site along with the patient-side adapter 426 and can be connected to the PoP node 1072 by the connection 1062. The patient-side adapter 426 can be connected to the gateway 429 via a suitable network connection available at the patient site, such as LAN. In some instances, one or more of the gateways 419 and 429 may not be present, and a respective adapter 418 or 426 can be directly connected to a respective PoP node 1070 or 1072 via a respective onramp connection 1060 or 1062.

The physician-side adapter 418 can be connected to the PoP 1070 (for instance, via the gateway 419) using the onramp connection 1060. The patient-side adapter 426 can be connected to the PoP 1072 (for instance, via the gateway 429) using the onaramp connection 1062. The onramp connections 1060 and 1062 can be suitable last mile connections. For example, any one or more of the onramp connections 1060 or 1062 can be a dedicated connection. A dedicated connection can be an exclusive connection that ensures consistent bandwidth and service quality (or, more generally, consistent performance). Alternatively or additionally, in some instances, any one or more of the onramp connections 1060 or 1062 can be a direct connection. A direct connection may not pass through any routers or switches to connect an adapter to a PoP. A direct connection can provide high performance (such as, high bandwidth, low latency, low packet loss, and low jitter). In some cases, any one or more of the onramp connections 1060 and 1062 can be a fiberoptic connection.

PoP 1070 and PoP 1072 can be connected via a backbone connection 1064. The backbone connection 1064 can be a direct connection, as described herein. Alternatively or additionally, in some instances, the backbone connection 1064 can be a dedicated connection, as described herein.

FIG. 6B can illustrate a scenario of the physician input console 416 being located far away from the robot 422 so that multiple PoPs 1070 and 1072 are involved in forming the connection that facilitates remote control of the robot 422.

In some cases, a single PoP can be used to connect a physician console to a robot. This can occur when the physician console and the robot are located close to each other. With reference to FIG. 6A, suppose that a physician located in San Francisco wishes to remotely perform a medical procedure on a patient located in Oakland. A physician console located in San Francisco, such as at the site 1034, would need to be connected to a robotic system located in Oakland, such as at the site 1030. The PoP 1014 located in Los Angeles is illustrated as being a single PoP that covers California. The PoP 1014 can be used to establish a connection between the physician console and the robot. Routing traffic between the physician console located in San Francisco and the robot located in Oakland through the PoP 1014 located in Los Angeles (and the onramps 1051 and 1054 respectively connecting the physician console and the robot to the PoP) can be more efficient than routing traffic via a shortest path connection outside the network 1000 between the physician console and the robot. Even though such shortest path connection spans a shorter distance than the connection through the PoP 1014 and the onramps 1051 and 1054, the connection through the PoP 1014 would be more desirable for the reasons explained herein. For instance, the shortest path connection would likely span the public Internet, which may not provide sufficient network performance (such as, high bandwidth, low latency, low packet loss, and low jitter) for remotely controlling a medical procedure.

As another example, FIG. 6E illustrates sites labeled “physician console site #1” and “physician console site #2” housing one or more physician consoles and connected to PoP 1 via onramp connections 1 and 2, respectively. Also illustrated is a site labeled “robot site #1” that houses one or more robotic systems and connected to PoP 1 via an onramp connection 3. Suppose that a physician operating a physician console located at the physician console site 1 wishes to remotely control a robotic system located at the robot site 1. To facilitate this, a first connection (illustrated as “connection #1”) traversing the onramp connection 1 and the onramp connection 3 would be established. This first connection traverses a single PoP node. Suppose further that a physician operating a physician console located at the physician console site 2 wishes to remotely control a robotic system located at a site labeled “robot site #2,” which is connected to PoP 2 via an onramp connection 4. To facilitate this, a second connection (illustrated as “connection #2”) traversing the onramp connection 2, a backbone connection connecting PoP 1 and PoP 2, and the onramp connection 4 would be established. As described herein, the second connection can be established and maintained simultaneously with and independent of the first connection.

A particular physician console or robotic system can be assigned to a PoP based on proximity. For instance, the closest PoP in the network 1000 can be assigned. With reference to FIG. 6A, all physician consoles and robots located at sites in California and Nevada can be assigned to the PoP 1014. This assignment may change in case the PoP 1014 goes down or experiences degradation in performance.

In some instances, the network 1000 can facilitate connection of multiple physician consoles to a single robotic system. Such arrangement can be advantageous for mentoring.

Advantageously, the network 1000 can provide efficiency, scalability, flexibility, and reliability. Anytime a new site housing one or more physician consoles or robots is being added to the network 1000, only the last mile connection to a suitable PoP may need to be arranged.

### Connection Testing

As described herein, a network connection of a physician-side adapter (such as, surgeon-side adapter) or a patient-side adapter can be tested with a simulated adapter or simulated console or robotic procedure system (such as, robotic surgery system). Testing can advantageously ensure that the network connection is ready to reliably support a remotely controlled robotic-assisted medical procedure session (such as, a remotely controlled robotic-assisted surgical session) prior to initiation of such session. Testing can be performed prior to establishing the session. In some cases, testing can be performed at the time of initialization of one or more of a surgeon-side adapter, patient-side adapter, surgeon console, or surgical robot (such as, during a power on or reboot). In some instances, testing can be performed periodically after the initialization.

With reference to FIG. 7, a remote robotic-assisted medical system 1400 (such as, a remote surgery system) can include one or more simulated (or mock) physician consoles 1416 (such as, surgeon consoles) and simulated (or mock) robotic procedure systems 1422 (such as, robotic surgery systems). The remote surgery system 1400 can include one or more real surgeon consoles and robotic surgery systems, which are illustrated in FIGS. 4 and 5. One or more of the simulated surgeon console 1416 or simulated robotic surgery system 1422 can be implemented as hardware, software and/or firmware being executed by one or more processors, or combination of the foregoing.

The simulated surgeon console 1416 can be similar to the surgeon console 416 illustrated in FIG. 5. As described herein, the simulated surgeon console 1416 can include a bridge 1502 (which can be implemented in software and/or firmware) that connects an interface 1512 with one or more of the orchestration application 432, connectivity server 434, or adapter management system 436. As described herein, the simulated surgeon console can receive image data 1520 from a real or simulated robotic surgery system, provide control signals 1530 to the real or simulated robotic surgery system, and exchange status information 1540 with the real or simulated robotic surgery system. In some instances, the bridge 1502 can function as a gateway, which can forward traffic between the interface 1512 and one or more of the orchestration application 432, connectivity server 434, or adapter management system 436.

The simulated robotic surgery system 1422 can be similar to the robotic surgery system 422 illustrated in FIG. 5. As described herein, the simulated robotic surgery system 1422 can include a bridge 1504 (which can be implemented in software and/or firmware) that connects an interface 1514 with one or more of the orchestration application 432, connectivity server 434, or adapter management system 436. As described herein, the simulated robotic surgery system can provide image data 1522 to a real or simulated surgeon console, receive control signals 1532 from the real or simulated robotic surgery system, and exchange status information 1542 with the real or simulated robotic surgery system. In some instances, the bridge 1504 can function as a gateway, which can forward traffic between the interface 1514 and one or more of the orchestration application 432, connectivity server 434, or adapter management system 436. While the network management system 435 is not illustrated in FIG. 7, the network management system can be present in the system 1400.

Testing of a network connection with a real surgeon console (such as, the surgeon console 416) can be performed using the simulated robotic surgery system 1422. Testing of the network connection can be initiated by a surgeon or another user or automatically (for instance, by a simulator being executed by the real surgeon console). Connection between the real surgeon console and the simulated robotic surgery system 1422 can be established by the real surgeon console or by the connectivity server 434 (using any of the approaches described herein). Testing of a network connection with a real robotic surgery system (such as, the surgical robot 422) can be performed using the simulated surgeon console 1416. Testing of the network connection can be initiated by a nurse or another user or automatically (for instance, by a simulator being executed by the real robotic surgery system). Connection between the real robotic surgery system and the simulated surgeon console 1416 can be established by the real robotic surgery system or by the connectivity server 434 (using any of the approaches described herein).

Testing can mimic transmission of data over a network connection. Testing can utilize the same payload size of messages or packets, frequency of transmission of messages or packets, and transmission protocol as would be used during a real robotic surgery session. Therefore one or more of 1520, 1530, 1540, 1522, 1532 or 1542 may be packets of random data simulating one or more of the size, frequency, or protocol of one or more of control signals, video data, or status data, while not actually being control signal data, video data, or status data. In some instances, transmission of control signals, image data, and status information can be performed (using any of the approaches described herein).

To increase the effectiveness of testing, the simulated robotic surgery system 1422 can be connected to the same PoP node to which the real robotic surgery system is (or would be) connected. The simulated surgeon console 1416 can be connected to the same PoP node to which the real surgeon console is (or would be connected).

### Switching Control

To enhance safety, a robotic-assisted medical system (such as, a robotic surgery system) can include the switching circuitry 140 illustrated in FIG. 2. The switching circuitry 140 can facilitate switching from local control over the connection 115 (such as, control shown in FIG. 1) to a remote control over the network 208 (such as, control shown in FIG. 2). Suppose that a remote surgeon 112 is performing a procedure using a surgeon input console 114 that is located remotely from the robotic surgery system 100. As described herein, the surgeon input console 114 can be connected to a robotic surgery system 100 via the network 208. The network 208 can be a WAN. In some instances, the network 208 can include one or more features of the network 1000 described herein. Suppose that during the procedure, one or more of the remote surgeon input console 114 or the network 208 experiences a malfunction. A local surgeon can take over control of the procedure using a locally-positioned surgeon input console 114 that is connected to the robotic surgery system 100 with via the connection 115. The connection 115 can be isolated from any other network (for instance, be a direct LAN).

The switching circuitry 140 can be used to efficiently facilitate such a change of control from remote to local (or vice versa). The switching circuitry 140 can operate in two modes. In a first mode (which may be a default mode), the switching circuitry 140 can allow only local control of the robotic surgery system 100 via a surgeon input console 114 connected via the connection 115. In a second mode, the switching circuitry 140 can allow only remote control of the robotic surgery system 100 via a surgeon input console 114 connected via the network 208. The switching circuitry can include a multiplexer circuitry configured to switch flow of control signals, image data, and status data between the connection 115 and the network 208.

The switching circuitry 140 can be at least partially exposed on a surface of the robotic surgery system 100 so that it can be quickly and easily manipulated by a nurse or another user. The switching circuitry 140 can include a switch or a button. In some instances, the switching circuitry can include a graphical user interface that, for instance, displays a switch, button, checkbox, sliding control, or the like. The graphical user interface can be displayed on a touch screen display. In some instances, the switching circuitry 140 can respond to a voice command to switch control. In some cases, the switching circuity 140 can be controlled remotely, such as, via a command transmitted over the network 208.

As another example, a local surgeon 112 can be performing a procedure and a remote surgeon 112 can monitor the procedure from a remote surgeon-side location. Suppose that the local surgeon 112 needs assistance with the procedure (for instance, the local surgeon can be a less experienced surgeon than the remote surgeon). The switching circuitry 140 can efficiently facilitate change of control from local to remote. After the remote surgeon has provided the necessary assistance, control can be switched back to local using the switching circuitry 140.

The switching circuitry 140 can be integrated with the robotic surgery system 100. This can encompass being wholly separate from the robotic surgery system 100 or being wholly or partially a portion of the robotic surgery system 100. In some implementations, the switching circuitry 140 can be implemented as software and/or firmware being executed by one or more processors of the robotic surgery system 100.

A switching circuity 142 can be similarly integrated with a surgeon input console 114. The switching circuitry 142 can be similar to the switching circuitry 140 and can function as described above. The switching circuitry 142 can be used alternatively or additionally to the switching circuitry 140.

### Allowing Control of Robotic Procedure System

Granting control of a patient-side robotic medical procedure system (such as, a patient-side robotic surgery system) to a physician (such as, a surgeon) operating a physician console (such as, surgeon console) directly implicates patient safety. When a surgeon is local to the patient-side robotic surgery system (such as, located in or near an operating room where the system and patient are placed), ensuring patient safety may be more straightforward than when the surgeon is located remotely. For example, in the local implementation, only one surgeon console would be connected to the patient-side robotic surgery system. In the remote example, in contrast, multiple surgeon consoles may be able to connect to the patient-side robotic surgery system, as described herein. Accordingly, the problem of ensuring patient safety prior to granting control is much more difficult to resolve in the remote surgery context. As described in this section, prior to granting control to the remote surgeon operating a remote surgeon console, various checks for controlling access to the patient-side robotic surgery system may need be performed to ensure that patient safety is not compromised.

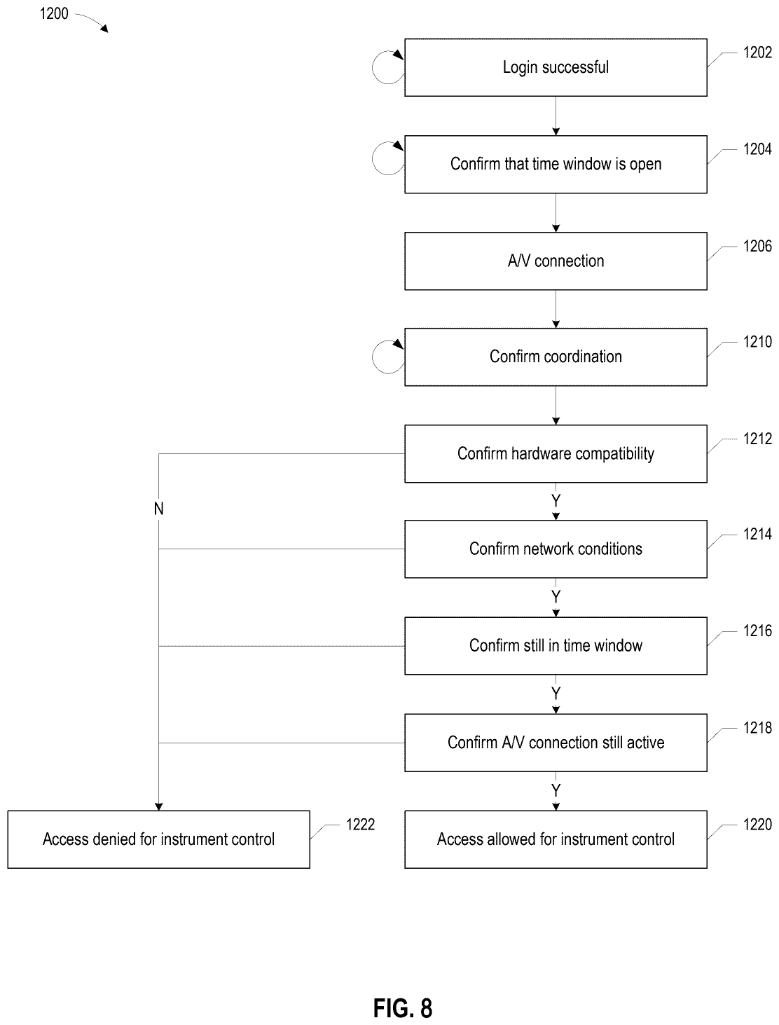

FIG. 8 illustrates a flow chart of a process 1200 for allowing to remotely control a patient-side robotic procedure system (such as, patient-side robotic surgical system). The process 1200 can be implemented by any of the remote medical procedure systems described herein, such as the system 400, 500, or 1400. In some cases, the process 1200 can be implemented, at least in part, by the orchestration application 432. The process 1200 can be performed as part of processing a request to establish a session between a surgeon console collocated with a surgeon and a patient-side robotic surgery system collocated with a patient to allow the surgeon to remotely control movement and actuation of at least one surgical instrument (and/or at least one imaging system) of the patient-side robotic surgery system and operate on the patient.

The process 1200 can start at step 1202, where a surgeon can login to obtain access for remotely controlling at least one surgical instrument of a patient-side robotic surgery system. For example, the surgeon can enter the surgeon's login credentials (e.g., username and password) into a user interface of an application being executed on the surgeon PC 412. The surgeon PC 412 can communicate with the orchestration application 432, as described herein. In some examples, the orchestration application 432 can perform authentication of the login credentials. In some instances, when the surgeon inputs login credentials that fail the authentication service, the surgeon may not granted access to control the patient-side robotic surgery system. When the login is unsuccessful, step 1202 can be configured to loop and have the surgeon enter (or re-enter) login credentials to attempt to gain access. In some examples, the surgeon may not be able to remotely operate on the patient without verification of the surgeon's credentials.

At step 1204, once the surgeon has selected the desired surgery and patient (for instance, on a schedule), the process 1200 can confirm that a time window for remotely operating on the patient is open. In some instances, when the process 1200 determines that the time window is not open, step 1210 can be configured to loop and check again whether the time window is open. The surgeon may not be allowed to remotely operate on the patient outside the time window.

A session for remotely controlling the patient-side surgical system can be associated with a time window during which a surgeon is allowed to remotely control a patient-side robotic system to operate on a patient, subsequent to a completion of coordination (such as, signoffs) as described herein. The time window can correspond to the scheduled time of the surgical session (such as, to an allotted session time slot). After such time window has opened and over duration of the time window, the operational connection from the surgeon's surgical console to remotely control the patient-side robotic system may be allowed, subsequent to the completion of coordination, while an operational connection from any other surgical console to remotely control the patient-side robotic system may be disallowed. After the time window has ended, an operational connection from the surgeon's surgical console to remotely control the patient-side robotic system may be disallowed as it is possible that another time window has been opened to permit the same or another surgical console to connect to the patient-side robotic system to remotely control the patient-side robotic system to operate on a different patient. That is, time windows for remotely controlling a particular patient-side robotic system can be arranged in non-overlapping chronological order. The operational connection may be initiated responsive to completion of the coordination between the patient side and surgeon side, which, as described herein, can involve completion of one or more checklists and attestation of the one or more checklists (this can be referred to as signoffs). The surgeon can be informed that the time window has been opened, for instance, via the surgeon PC 412 and the monitor 414. In response to the notification, the surgeon can initiate the operational connection to remotely control the patient-side robotic surgery system. On the patient side, the operating room staff can be informed that the time windows have been opened, for instance, via the nurse PC 420.

At step 1206, the process 1200 can wait for or establish an A/V connection between the surgeon side and the patient side. As described herein, the A/V connection can be established between the surgeon PC 412 and the telemedicine device 428 to facilitate provision of an audio and/or video feed from the patient site 404 to the surgeon site 402 (and vice versa) as well as facilitate a two-way audio and/or video communication between the patient site 404 and the surgeon site 402. The A/V connection can allow the surgeon and the operating room staff to communicate in real-time. In some examples, the surgeon may not be allowed to remotely operate on the patient without the A/V connection having been established.

At step 1210, the process 1200 can perform coordination between the patient side and the surgeon side. As described herein, coordination can generally refer to a handshake protocol being performed between the patient side and the surgeon side to ensure that both sides are ready for execution of a remotely controlled surgical procedure. As an example, coordination can involve completion of a checklist on the patient side and attestation of the checklist by the surgeon. The process 1200 can remain at step 1210 until coordination has been completed. In some examples, the surgeon may not be allowed to remotely to remotely operate on the patient until completion of coordination.

At step 1212, the process 1200 can verify compatibility between the patient-side surgical system and a surgeon console of the surgeon, as described herein. If compatibility is not verified, the process 1200 can transition to step 1222 where access to the patient-side robotic surgery system is denied.

If compatibility is verified, the process can transition to step 1214 where the process 1200 can confirm network conditions. One or more networks connecting the surgeon console to the patient-side surgery system (such as, 208 or 430) can be selected and configured to reduce transmission delay and increase reliability and stability in order to remotely control the surgical procedure. Prior to allowing access to the patient-side robotic surgery system, the process 1200 (for instance, via the connectivity server 434) can verify that one or more networks connecting the surgeon console and the patient-side robotic surgery system meet network conditions (or network requirements) for remotely controlling the surgical procedure. In some examples, the process 1200 can perform one or more network tests to verify that the network conditions are satisfied. For instance, the connectivity server 434 may perform automated network tests on each endpoint on a cadence of, for example, every 30 minutes. The orchestration application 432 can query the connectivity server 434 to report the results of the most recent network test, or to initiate performance of a new network test. The connectivity server 434 can then analyze the one or more results of the network test to assess a quality of the connection. In some examples, the orchestration application 432 can perform the network test without involving the connectivity server 434.

In some examples, the network conditions can include one or more of network latency, network jitter, network packet loss, and network bandwidth. At step 1214, the process 1200 can assess whether the network conditions satisfy one or more threshold values. In some cases, the process 1200 can assess whether at least one, at least two, or at least three of the foregoing conditions satisfy the respective threshold values. The process 1200 can verify that network latency satisfies a latency threshold, such as a value between about 1-2 ms and about 500 ms (such as, about 300 ms). Network latency verification can ensure that one or more networks are capable of reliably and quickly transferring image data of the surgical site (such as, video) from the patient side to the surgeon side, such that the surgeon can respond in a safe and timely manner to the image data. The process 1200 can assess whether network jitter satisfies a jitter threshold, such as below about 1 ms or below about 10 ms. Since network jitter causes variability in the latency of transmitted packets, it can affect the timing and order of control signals and image data. By verifying that network jitter does not exceed the jitter threshold, the process 1200 can ensure the reliable and prompt transfer of control signals and image data. The process 1200 can assess whether network packet loss satisfies a packet loss threshold, such as about 1% or about 5%. Packet loss verification can ensure that one or more networks are capable of reliably and quickly transferring control signals and image data. The process 1200 can assess whether network bandwidth satisfies a bandwidth threshold, such as at least about 1 Gigabit/second (Gb/s). Network bandwidth verification can ensure that one or more networks are capable of reliably and quickly transferring control signals and image data.

The one or more networks can include one or more primary and one or more secondary networks to provide for backup and/or redundant transmission of data between the surgeon side and the patient side. This can further facilitate robustness and reliability. In some examples, in step 1214, the process 1200 can confirm that a primary network connection satisfies the network conditions and that at least one secondary network connection is available. In some cases, the process 1200 can confirm in step 1214 that the at least one secondary network also satisfies at least some of the network conditions (or, in some cases, all of the network conditions).

If the network conditions are not verified, the process 1200 can transition to step 1222 where access to the patient-side robotic surgery system is denied.

If the network conditions are verified, the process 1200 can transition to step 1216 where the process 1200 can confirm the time window remains open. If not, the process 1200 can transition to step 1222 where access to the patient-side robotic surgery system is denied.

If it is verified that the time window remains open, the process 1200 can transition to step 1218, where the process 1200 can confirm that the A/V connection remains active, to ensure the surgeon can maintain a dialog with the operating room staff during the surgical procedure. If not, the process 1200 can transition to step 1222 where access to the patient-side robotic surgery system is denied.

If it is verified that the A/V connection remains active, the process 1200 can transition to step 1220, where the process 1200 can grant access for remotely controlling at least one surgical instrument of the patient-side robotic surgery system. In some instances, the process 1200 can establish the session between the surgeon console and the patient-side robotic surgery system.

As described herein, at step 1222, the process 1200 can deny access for remotely controlling the patient-side robotic surgery system. For example, the process 1200 may block establishment of the session between the surgeon console and the patient-side robotic surgery system. The steps of the process 1200 can be executed in the illustrated order or in a different order. In some instances, one or more of the steps of the process 1200 can be optional and independent with respect to one another. In some instances, individual steps discussed above can be removed from the process 1200. In some instances, the process 1200 can include one or more additional or substitute verification tasks. For example, the process 1200 can perform one or more of: confirm surgical consent, confirm that a surgeon-side adapter (and/or the surgeon console) and a patient-side adapter (and/or the patient-side robotic surgery system) are in good health, verify a patient's identity, confirm a surgeon's licensing, provisioning, and certification, confirm the surgical procedure, confirm the surgeon completed a pre-operation visit, confirm the surgeon has a business relationship with patient-side site, or the like.

Surgical consent can be a form that the patient signed prior to the operation. For example, the patient can sign (for instance, physically or electronically) a consent form and the patient's consent can be recorded by the process 1200. In some cases, the process 1200 can present a user interface to provide the patient with an electronic surgical consent form and accept the patient's signature. In some instances, the process 1200 can verify surgical consent satisfied prior to granting access to remotely control the patient-side robotic surgery system.

Confirmation that the surgeon-side adapter (or, more generally, the surgeon console) and the patient-side adapter (or, more generally, the patient-side robotic surgery system) are in good health can include monitoring status information (such as, heartbeat signals or the like) transmitted by the respective adapters (or, more generally, by the surgeon console and the patient-side robotic surgery system). For example, status monitoring can be performed by the adapter management system 436. The adapter management system 436 can transmit status information to the orchestration application 432. In some examples, the orchestration application 432 can query the adapter management system 436 to for status information. In response, the adapter management system 436 can provide received status information or ping the respective adapters (or, more generally, the surgeon console and the patient-side robotic surgery system) for status information. Based on the status information, the orchestration application 432 can assess whether the health of the respective adapters (or, more generally, the surgeon console and the patient-side robotic surgery system) is satisfactory for remote surgery. In some examples, access to the patient-side robotic surgery system can be restricted if the status of any of the adapters (or, more generally, of the surgeon console or the patient-side robotic surgery system) indicates bad health (for instance, the reported status indicates an error or no status has been reported).

Verification of the patient identity can include comparing the patient in the operating room to the patient listed in the schedule. Such verification can be part of the coordination in step 1210. In some examples, the orchestration application 432 can prompt medical personnel in the operating room to confirm the identity of the patient prior to the surgeon gaining access to the patient-side robotic surgery system. Medical personnel can provide confirmation of the patient with the orchestration application 432, such as via a user interface (for instance, a checkbox), verbally (for instance, via the A/V connection or using a telephone), or the like. In some examples, verification of the patient identity can include verifying the patient's biometric information. For example, the verification can include a comparison of a fingerprint scan of the patient at the time of operation with a recorded fingerprint of the patient prior to the time of operation. The orchestration application 432 can be configured to receive the fingerprint scan and compare the scan with the recorded fingerprint of the patient. Access may be granted to the surgeon only in response to the comparison resulting in a positive match between the fingerprints. Other biometric information can be used in a similar manner to verify the identity of the patient.

Verification procedure for granting access to control the patient-side robotic surgery system can include checking that the surgery being performed is the surgery scheduled for the patient. For example, the orchestration application 432 can prompt medical personnel in the operating room to confirm the surgical procedure is the scheduled surgical procedure. This can be performed using any of the approaches described herein, such as, via a user interface, vocally, or the like. Access may be granted only in response to a match between the surgical procedures.

Verification procedure for granting access can include confirming the surgeon's professional credentials, such as one or more of licensing, provisioning, and certification. The professional credentials can indicate permission to perform particular type of surgery, right to perform surgery at a particular hospital or medical center, or the like. Verification procedure for granting access can include confirmation that the surgeon completed a pre-operation visit and confirmation that the surgeon has a business relationship with the particular hospital or medical center. These verifications can be performed using any of the approaches described herein, such as, via a user interface, vocally, or the like.

Providing access for controlling the patient-side robotic surgery system can involve transmission of control signals for moving and actuating at least one instrument of the patient-side robotic surgery system. Prior to transmission of such control signals, it may be necessary to establish the A/V connection that provides a two-way audio and/or video communication between the patient side and the surgeon side as well as well as arrange for transmission of image data of the surgical site to the surgeon side. It may be unsafe to allow the surgeon to operate on the patient if the surgeon were not able to have a dialog with the patient-side surgical staff via the A/V connection. For example, the surgical staff may verbally communicate a patient-side issue that would require the remote surgeon to discontinue or alter the operation of one or more instruments. Additionally, it can be advantageous for the surgeon to communicate with the surgical staff prior to initiating the surgical procedure in order to talk through coordination or the like. Further, it may be unsafe to allow the surgeon to operate one or more instruments if the surgeon were not seeing the live results of that operation via the transmitted image data of the surgical site.

The process 1200 may grant access for remotely controlling at least one surgical instrument of the patient-side robotic surgery system after it has verified that 1) the A/V connection has been established and 2) the image data is ready to be transmitted or is being transmitted. In some implementations, the A/V connection can be established prior to commencement of transmission of the image data or substantially simultaneously with commencement of transmission of the image data (which can encompass a time period after commencement of transmission of the image data). For example, the A/V connection can be established within 2 seconds of commencement of transmission of the image data (such as, up to 2 seconds after commencement of transmission of the image data). In certain cases, control signals may be transmitted after transmission of the image data has commenced or substantially simultaneously with transmission of the image data (which can encompass a time period prior to commencement of transmission of the image data). For example, commencement of transmission of the control data can be within 500 ms of commencement of transmission of the image data (such as, up to 500 ms before commencement of transmission of the image data).

### Example Implementations

Examples of the implementations of the present disclosure can be described in view of the following example clauses. The features recited in the below example implementations can be combined with additional features disclosed herein. Furthermore, additional inventive combinations of features are disclosed herein, which are not specifically recited in the below example implementations, and which do not include the same features as the specific implementations below. For sake of brevity, the below example implementations do not identify every inventive aspect of this disclosure. The below example implementations are not intended to identify key features or essential features of any subject matter described herein. Any of the example clauses below, or any features of the example clauses, can be combined with any one or more other example clauses, or features of the example clauses or other features of the present disclosure.

Clause 1. A system for remotely controlling robotic assisted medical procedures, the system including a plurality of point-of-presence (PoP) nodes configured to connect any physician console of a plurality of physician consoles to any compatible patient-side robotic procedure system of a plurality of patient-side robotic procedure systems configured to be located remotely from the plurality of physician consoles. The system can include a first PoP node of the plurality of PoP nodes configured to be connected to a first physician console of the plurality of physician consoles by a first dedicated connection and a second physician console of the plurality of physician consoles by a second dedicated connection. The system can include a second PoP node of the plurality of PoP nodes configured to be connected to a first patient-side robotic procedure system of the plurality of patient-side robotic procedure systems by a third dedicated connection and a second patient-side robotic procedure system of the plurality of patient-side robotic procedure systems by a fourth dedicated connection. The second PoP node can be configured to be connected to the first PoP node by a fifth connection. The first and second PoP nodes can be configured to enable a first session connection between the first physician console and the first patient-side robotic procedure system to facilitate performing a first robotic assisted medical procedure. The first session connection can span the first dedicated connection connecting the first physician console to the first PoP node, the third dedicated connection connecting the first patient-side robotic procedure system to the second PoP node, and the fifth connection connecting the first and second PoP nodes. The first and second PoP nodes can be configured to, via the first session connection, facilitate transmission of first control data from the first physician console to the first patient-side robotic procedure system, the first control data being used by the first patient-side robotic procedure system to control at least one instrument or at least one imaging system of the first patient-side robotic procedure system. The first and second PoP nodes can be configured to, via the first session connection, facilitate transmission of first image data obtained by the at least one imaging system of the first patient-side robotic procedure system to the first physician console; enable a second session connection between the second physician console and the second patient-side robotic procedure system to facilitate performing a second robotic assisted medical procedure. The second session connection can span the second dedicated connection connecting the second physician console to the first PoP node, the fourth dedicated connection connecting the second patient-side robotic procedure system to the second PoP node, and the fifth connection connecting the first and second PoP nodes. The first and second PoP nodes can be configured to, via the second session connection, facilitate transmission of second control data from the second physician console to the second patient-side robotic procedure system, the second control data being used by the second patient-side robotic procedure system to control at least one instrument or at least one imaging system of the second patient-side robotic procedure system; and via the second session connection, facilitate transmission of second image data obtained by the at least one imaging system of the second patient-side robotic procedure system to the second physician console.

Clause 2. The system of clause 1, wherein the first and second session connections facilitate simultaneous transmission of first data over the first session connection and second data over the second session connection such that the first and second robotic assisted medical procedures can be performed simultaneously, and wherein the first data is separated from the second data.

Clause 3. The system of any one of clauses 1 to 2, wherein the at least one of first, second, third, or fourth dedicated connections further includes a direct connection.

Clause 4. The system of any one of clauses 1 to 3, wherein the at least one of first, second, third, or fourth dedicated connections includes a leased line.

Clause 5. The system of any one of clauses 1 to 4, wherein the fifth connection includes a dedicated connection.

Clause 6. The system of any one of clauses 1 to 5, wherein at least one of the first or second PoP nodes is configured to be connected to at least one other PoP node.

Clause 7. The system of any one of clauses 1 to 6 can include a non-transitory computer readable medium storing instructions that, when executed by at least one processor, cause the at least one processor to: receive a first request to establish the first session connection between the first physician console and the first patient-side robotic procedure system; responsive to receiving the first request, establish the first session connection between the first physician console and the first patient-side robotic procedure system; receive a second request to establish the second session connection between the second physician console and the second patient-side robotic procedure system; and responsive to receiving the second request, establish the second session connection between the second physician console and second first patient-side robotic procedure system.

Clause 8. A system for remotely controlling robotic assisted medical procedures, the system including a plurality of point-of-presence (PoP) nodes configured to connect any physician console of a plurality of physician consoles to any compatible patient-side robotic procedure system of a plurality of patient-side robotic procedure systems configured to be located remotely from the plurality of physician consoles. The system can include a first PoP node of the plurality of PoP nodes configured to be connected to a first physician console of the plurality of physician consoles by a first dedicated connection. The system can include a second PoP node of the plurality of PoP nodes configured to be connected to a first patient-side robotic procedure system of the plurality of patient-side robotic procedure systems by a second dedicated connection, the second PoP node configured to be connected to the first PoP node by a third connection. The system can include a third PoP node of the plurality of PoP nodes configured to be connected to the first PoP node by a fourth connection and the second PoP node by a fifth connection. The first, second, and third PoP nodes can be configured to enable a first session connection between the first physician console and the first patient-side robotic procedure system to facilitate performing a first robotic assisted medical procedure, the first session connection spanning the first dedicated connection connecting the first physician console to the first PoP node, the second dedicated connection connecting the first patient-side robotic procedure system to the second PoP node, and one of 1) the third connection connecting the first and second PoP nodes or 2) the fourth connection connecting the first PoP node to the third PoP node and the fifth connection connecting the second PoP node to the third PoP node. The first, second, and third PoP nodes can be configured to, via the first session connection, facilitate transmission of first control data from the first physician console to the first patient-side robotic procedure system, the first control data being used by the first patient-side robotic procedure system to control at least one instrument or at least one imaging system of the first patient-side robotic procedure system. The first, second, and third PoP nodes can be configured to, via the first session connection, facilitate transmission of first image data obtained by the at least one imaging system of the first patient-side robotic procedure system to the first physician console.

Clause 9. The system of clause 8, wherein the first, second, and third PoP nodes can be configured to, at a first time, enable the first session connection that spans the first dedicated connection connecting the first physician console to the first PoP node, the second dedicated connection connecting the first patient-side robotic procedure system to the second PoP node, and the third connection connecting the first and second PoP nodes. The first, second, and third PoP nodes can be configured to, at a second time subsequent to the first time, determine that performance of the third connection satisfies a degradation threshold and in response to determining that performance of the third connection satisfies the degradation threshold, adjust the first session connection to span the first dedicated connection, the second dedicated connection, the fourth connection connecting the first PoP node to the third PoP node, and the fifth connection connecting the second PoP node to the third PoP node.

Clause 10. The system of any one of clauses 8 to 9, wherein the first PoP node of the plurality of PoP nodes can be configured to be connected to a second physician console of the plurality of physician consoles by the first dedicated connection. The second PoP node of the plurality of PoP nodes can be configured to be connected to a second patient-side robotic procedure system of the plurality of patient-side robotic procedure systems by the second dedicated connection. The first, second, and third PoP nodes can be configured to enable a second session connection between the second physician console and the second patient-side robotic procedure system to facilitate performing a second robotic assisted medical procedure, the second session connection spanning the first dedicated connection connecting the second physician console to the first PoP node, the second dedicated connection connecting the second patient-side robotic procedure system to the second PoP node, and one of 1) the third connection connecting the first and second PoP nodes or 2) the fourth connection connecting the first PoP node to the third PoP node and the fifth connection connecting the second PoP node to the third PoP node. The first, second, and third PoP nodes can be configured to, via the second session connection, facilitate transmission of second control data from the second physician console to the second patient-side robotic procedure system, the second control data being used by the second patient-side robotic procedure system to control at least one instrument or at least one imaging system of the second patient-side robotic procedure system, The first, second, and third PoP nodes can be configured to, via the second session connection, facilitate transmission of second image data obtained by the at least one imaging system of the second patient-side robotic procedure system to the second physician console.

Clause 11. The system of clause 10, wherein the first and second session connections facilitate simultaneous transmission of first data over the first session connection and second data over the second session connection such that the first and second robotic assisted medical procedures can be performed simultaneously, and wherein the first data is separated from the second data

Clause 12. The system of any one of clauses 8 to 11, wherein the third PoP node of the plurality of PoP nodes can be configured to be connected to a third patient-side robotic procedure system of the plurality of patient-side robotic procedure systems by a sixth dedicated connection. The first and third PoP nodes can be configured to enable a third session connection between the first physician console and the third patient-side robotic procedure system to facilitate performing a third robotic assisted medical procedure, the third session connection spanning the first dedicated connection connecting the first physician console to the first PoP node, the sixth dedicated connection connecting the third patient-side robotic procedure system to the third PoP node, and the fourth connection connecting the first PoP node to the third PoP node. The first and third PoP nodes can be configured to, via the third session connection, facilitate transmission of third control data from the first physician console to the third patient-side robotic procedure system, the third control data being used by the third patient-side robotic procedure system to control at least one instrument or at least one imaging system of the third patient-side robotic procedure system. The first and third PoP nodes can be configured to, via the third session connection, facilitate transmission of third image data obtained by the at least one imaging system of the third patient-side robotic procedure system to the first physician console.

Clause 13. The system of any one of clauses 8 to 12, wherein the at least one of first or second dedicated connections further includes a direct connection.

Clause 14. The system of any one of clauses 8 to 13, wherein the at least one of first or second dedicated connections includes a leased line.

Clause 15. The system of any one of clauses 8 to 14, wherein at least one of the third, fourth, or fifth connections includes a dedicated connection.

Clause 16. The system of any one of clauses 8 to 15 can include a non-transitory computer readable medium storing instructions that, when executed by at least one processor, cause the at least one processor to: receive a first request to establish the first session connection between the first physician console and the first patient-side robotic procedure system; and responsive to receiving the first request, establish the first session connection between the first physician console and the first patient-side robotic procedure system.

Clause 17. A system for remotely controlling robotic assisted medical procedures, the system including a plurality of point-of-presence (PoP) nodes configured to connect any physician console of a plurality of physician consoles to any compatible patient-side robotic procedure system of a plurality of patient-side robotic procedure systems configured to be located remotely from the plurality of physician consoles. The system can include a first PoP node of the plurality of PoP nodes configured to be connected to a first physician console of the plurality of physician consoles by a first dedicated connection and a second physician console of the plurality of physician consoles by a second dedicated connection. The system can include a second PoP node of the plurality of PoP nodes configured to be connected to: a first patient-side robotic procedure system of the plurality of patient-side robotic procedure systems by a third dedicated connection. The system can include a second patient-side robotic procedure system of the plurality of patient-side robotic procedure systems configured to be connected to the first PoP node by a fourth dedicated connection. The second POP node can be configured to be connected to the first PoP node by a fifth connection. The first and second PoP nodes can be configured to enable a first session connection between the first physician console and the first patient-side robotic procedure system to facilitate performing a first robotic assisted medical procedure, the first session connection spanning the first dedicated connection connecting the first physician console to the first PoP node, the third dedicated connection connecting the first patient-side robotic procedure system to the second PoP node, and the fifth connection connecting the first and second PoP nodes. The first and second PoP nodes can be configured to, via the first session connection, facilitate transmission of first control data from the first physician console to the first patient-side robotic procedure system, the first control data being used by the first patient- side robotic procedure system to control at least one instrument or at least one imaging system of the first patient-side robotic procedure system. The first and second PoP nodes can be configured to, via the first session connection, facilitate transmission of first image data obtained by the at least one imaging system of the first patient-side robotic procedure system to the first physician console. The first and second PoP nodes can be configured to enable a second session connection between the second physician console and the second patient-side robotic procedure system to facilitate performing a second robotic assisted medical procedure, the second session connection spanning the second dedicated connection connecting the second physician console to the first PoP node and the fourth dedicated connection connecting the second patient-side robotic procedure system to the first PoP node. The first and second PoP nodes can be configured to, via the second session connection, facilitate transmission of second control data from the second physician console to the second patient-side robotic procedure system, the second control data being used by the second patient-side robotic procedure system to control at least one instrument or at least one imaging system of the second patient-side robotic procedure system. The first and second PoP nodes can be configured to, via the second session connection, facilitate transmission of second image data obtained by the at least one imaging system of the second patient-side robotic procedure system to the second physician console.

Clause 18. The system of clause 17, wherein the first and second session connections facilitate simultaneous transmission of first data over the first session connection and second data over the second session connection such that the first and second robotic assisted medical procedures can be performed simultaneously, and wherein the first data is separated from the second data

Clause 19. The system of any one of clauses 17 to 18, wherein the at least one of first or second dedicated connections further includes a direct connection.

Clause 20. The system of any one of clauses 17 to 19, wherein the at least one of first or second dedicated connections includes a leased line.

Clause 21. The system of any one of clauses 17 to 20, wherein the third connection includes a dedicated connection.

Clause 22. The system of any one of clauses 17 to 21 can include a non-transitory computer readable medium storing instructions that, when executed by at least one processor, cause the at least one processor to: receive a first request to establish the first session connection between the first physician console and the first patient-side robotic procedure system; responsive to receiving the first request, establish the first session connection between the first physician console and the first patient-side robotic procedure system; receive a second request to establish the second session connection between the second physician console and the second patient-side robotic procedure system; and responsive to receiving the second request, establish the second session connection between the second physician console and second first patient-side robotic procedure system.

### Other Variations

Any of the approaches described herein can utilize any of the examples disclosed in U.S. patents application Ser. Nos. 18/488,843 (now U.S. Pat. No. 12,089,906), 18/488844, 18/488886, each filed on Oct. 17, 2023, and each of which is incorporated by reference in its entirety. Any of the approaches described herein can utilize any of the examples disclosed in U.S. patents application Ser. Nos. 18/541,639 (now U.S. Pat. No. 12,042,239), 18/541673 (now U.S. Pat. No. 12,133,705), 18/541688 (now U.S. Pat. No. 12,064,202), each filed on Dec. 15, 2023, and each of which incorporated by reference in its entirety.

While certain examples are described in the context of remotely controlling a surgical procedure, the approaches described herein can be used for remotely controlling a variety of medical procedures, including invasive and non-invasive procedures as well as surgical and non-surgical procedures. The approaches described herein can be used for remotely controlling surgical procedures, interventional procedures, or diagnostic procedures (such as, procedures not involving manipulation of tissue).

While displaying has been used to describe certain examples of outputting information, any type of visual, auditory, or tactile output can be performed in addition to or alternatively.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, implementation, or example are to be understood to be applicable to any other aspect, implementation, or example described herein unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing implementations. The protection extends to any novel one, or any novel combination, of the features disclosed in this specification (including any accompanying claims, abstract and drawings), or to any novel one, or any novel combination, of the steps of any method or process so disclosed.

While certain implementations have been described, these implementations have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some cases, the actual steps taken in the processes illustrated and/or disclosed may differ from those shown in the figures. Depending on the implementation, certain of the steps described above may be removed, others may be added. For example, the actual steps and/or order of steps taken in the disclosed processes may differ from those shown in the figure. Various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific examples disclosed above may be combined in different ways to form additional implementations, all of which fall within the scope of the present disclosure.

Conditional language used herein, such as, among others, “can,” “could”, “might,” “may,” “e.g.,” and the like, unless specifically stated otherwise, or otherwise understood within the context as used, is generally intended to convey that certain implementation include, while other implementations do not include, certain features, elements and/or states. Thus, such conditional language is not generally intended to imply that features, elements and/or states are in any way required for one or more implementations or that one or more implementations necessarily include logic for deciding, with or without author input or prompting, whether these features, elements and/or states are included or are to be performed in any particular implementation. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain implementations require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, or within less than 0.01% of the stated value.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

While specific implementations have been described and illustrated, such implementations should be considered illustrative only and not as limiting. Accordingly, the scope of the present disclosure is not intended to be limited by the specific disclosures of preferred implementations herein, and may be defined by claims as presented herein or as presented in the future.

1. A system for remotely controlling robotic assisted medical procedures, the system comprising:
a plurality of point-of-presence (PoP) nodes configured to connect any physician console of a plurality of physician consoles to any compatible patient-side robotic procedure system of a plurality of patient-side robotic procedure systems configured to be located remotely from the plurality of physician consoles;
a first PoP node of the plurality of PoP nodes configured to be connected to: a first physician console of the plurality of physician consoles by a first dedicated connection and a second physician console of the plurality of physician consoles by a second dedicated connection; and
a second PoP node of the plurality of PoP nodes configured to be connected to: a first patient-side robotic procedure system of the plurality of patient-side robotic procedure systems by a third dedicated connection and a second patient-side robotic procedure system of the plurality of patient-side robotic procedure systems by a fourth dedicated connection, the second PoP node configured to be connected to the first PoP node by a fifth connection,
the first and second PoP nodes being further configured to:
enable a first session connection between the first physician console and the first patient-side robotic procedure system to facilitate performing a first robotic assisted medical procedure, the first session connection spanning the first dedicated connection connecting the first physician console to the first PoP node, the third dedicated connection connecting the first patient-side robotic procedure system to the second PoP node, and the fifth connection connecting the first and second PoP nodes;
via the first session connection, facilitate transmission of first control data from the first physician console to the first patient-side robotic procedure system, the first control data being used by the first patient-side robotic procedure system to control at least one instrument or at least one imaging system of the first patient-side robotic procedure system;
via the first session connection, facilitate transmission of first image data obtained by the at least one imaging system of the first patient-side robotic procedure system to the
enable a second session connection between the second physician console and the second patient-side robotic procedure system to facilitate performing a second robotic assisted medical procedure, the second session connection spanning the second dedicated connection connecting the second physician console to the first PoP node, the fourth dedicated connection connecting the second patient-side robotic procedure system to the second PoP node, and the fifth connection connecting the first and second PoP nodes;
via the second session connection, facilitate transmission of second control data from the second physician console to the second patient-side robotic procedure system, the second control data being used by the second patient-side robotic procedure system to control at least one instrument or at least one imaging system of the second patient-side robotic procedure system; and
via the second session connection, facilitate transmission of second image data obtained by the at least one imaging system of the second patient-side robotic procedure system to the second physician console.

2. The system of claim 1, wherein the first and second session connections facilitate simultaneous transmission of first data over the first session connection and second data over the second session connection such that the first and second robotic assisted medical procedures can be performed simultaneously, and wherein the first data is separated from the second data.

3. The system of claim 1, wherein the at least one of first, second, third, or fourth dedicated connections further comprises a direct connection.

4. The system of claim 1, wherein the fifth connection comprises a dedicated connection.

5. The system of claim 1, wherein at least one of the first or second PoP nodes is configured to be connected to at least one other PoP node.

6. The system of claim 1, further comprising a non-transitory computer readable medium storing instructions that, when executed by at least one processor, cause the at least one processor to:
receive a first request to establish the first session connection between the first physician console and the first patient-side robotic procedure system;
responsive to receiving the first request, establish the first session connection between the first physician console and the first patient-side robotic procedure system;
receive a second request to establish the second session connection between the second physician console and the second patient-side robotic procedure system; and
responsive to receiving the second request, establish the second session connection between the second physician console and second first patient-side robotic procedure system.

7. A system for remotely controlling robotic assisted medical procedures, the system comprising:
a plurality of point-of-presence (PoP) nodes configured to connect any physician console of a plurality of physician consoles to any compatible patient-side robotic procedure system of a plurality of patient-side robotic procedure systems configured to be located remotely from the plurality of physician consoles;
a first PoP node of the plurality of PoP nodes configured to be connected to a first physician console of the plurality of physician consoles by a first dedicated connection;
a second PoP node of the plurality of PoP nodes configured to be connected to a first patient-side robotic procedure system of the plurality of patient-side robotic procedure systems by a second dedicated connection, the second PoP node configured to be connected to the first PoP node by a third connection; and
a third PoP node of the plurality of PoP nodes configured to be connected to: the first PoP node by a fourth connection and the second PoP node by a fifth connection,
the first, second, and third PoP nodes being further configured to:
enable a first session connection between the first physician console and the first patient-side robotic procedure system to facilitate performing a first robotic assisted medical procedure, the first session connection spanning the first dedicated connection connecting the first physician console to the first PoP node, the second dedicated connection connecting the first patient-side robotic procedure system to the second PoP node, and one of 1) the third connection connecting the first and second PoP nodes or 2) the fourth connection connecting the first PoP node to the third PoP node and the fifth connection connecting the second PoP node to the third PoP node;
via the first session connection, facilitate transmission of first control data from the first physician console to the first patient-side robotic procedure system, the first control data being used by the first patient-side robotic procedure system to control at least one instrument or at least one imaging system of the first patient-side robotic procedure system; and
via the first session connection, facilitate transmission of first image data obtained by the at least one imaging system of the first patient-side robotic procedure system to the first physician console.

8. The system of claim 7, wherein the first, second, and third PoP nodes are configured to:
at a first time, enable the first session connection that spans the first dedicated connection connecting the first physician console to the first PoP node, the second dedicated connection connecting the first patient-side robotic procedure system to the second PoP node, and the third connection connecting the first and second PoP nodes; and
at a second time subsequent to the first time:
determine that performance of the third connection satisfies a degradation threshold; and
in response to determining that performance of the third connection satisfies the degradation threshold, adjust the first session connection to span the first dedicated connection, the second dedicated connection, the fourth connection connecting the first PoP node to the third PoP node, and the fifth connection connecting the second PoP node to the third PoP node.

9. The system of claim 7, wherein:
the first PoP node of the plurality of PoP nodes is further configured to be connected to a second physician console of the plurality of physician consoles by the first dedicated connection;
the second PoP node of the plurality of PoP nodes is further configured to be connected to a second patient-side robotic procedure system of the plurality of patient-side robotic procedure systems by the second dedicated connection; and
the first, second, and third PoP nodes are further configured to:
enable a second session connection between the second physician console and the second patient-side robotic procedure system to facilitate performing a second robotic assisted medical procedure, the second session connection spanning the first dedicated connection connecting the second physician console to the first PoP node, the second dedicated connection connecting the second patient-side robotic procedure system to the second PoP node, and one of 1) the third connection connecting the first and second PoP nodes or 2) the fourth connection connecting the first PoP node to the third PoP node and the fifth connection connecting the second PoP node to the third PoP node;
via the second session connection, facilitate transmission of second control data from the second physician console to the second patient-side robotic procedure system, the second control data being used by the second patient-side robotic procedure system to control at least one instrument or at least one imaging system of the second patient-side robotic procedure system; and
via the second session connection, facilitate transmission of second image data obtained by the at least one imaging system of the second patient-side robotic procedure system to the second physician console.

10. The system of claim 9, wherein the first and second session connections facilitate simultaneous transmission of first data over the first session connection and second data over the second session connection such that the first and second robotic assisted medical procedures can be performed simultaneously, and wherein the first data is separated from the second data.

11. The system of claim 7, wherein:
the third PoP node of the plurality of PoP nodes is further configured to be connected to a third patient-side robotic procedure system of the plurality of patient-side robotic procedure systems by a sixth dedicated connection; and
the first and third PoP nodes are further configured to:
enable a third session connection between the first physician console and the third patient-side robotic procedure system to facilitate performing a third robotic assisted medical procedure, the third session connection spanning the first dedicated connection connecting the first physician console to the first PoP node, the sixth dedicated connection connecting the third patient-side robotic procedure system to the third PoP node, and the fourth connection connecting the first PoP node to the third PoP node;
via the third session connection, facilitate transmission of third control data from the first physician console to the third patient-side robotic procedure system, the third control data being used by the third patient-side robotic procedure system to control at least one instrument or at least one imaging system of the third patient-side robotic procedure system; and
via the third session connection, facilitate transmission of third image data obtained by the at least one imaging system of the third patient-side robotic procedure system to the first physician console.

12. The system of claim 7, wherein the at least one of first or second dedicated connections further comprises a direct connection.

13. The system of claim 7, wherein at least one of the third, fourth, or fifth connections comprises a dedicated connection.

14. The system of claim 7, further comprising a non-transitory computer readable medium storing instructions that, when executed by at least one processor, cause the at least one processor to:
receive a first request to establish the first session connection between the first physician console and the first patient-side robotic procedure system; and
responsive to receiving the first request, establish the first session connection between the first physician console and the first patient-side robotic procedure system.

15. A system for remotely controlling robotic assisted medical procedures, the system comprising:
a plurality of point-of-presence (PoP) nodes configured to connect any physician console of a plurality of physician consoles to any compatible patient-side robotic procedure system of a plurality of patient-side robotic procedure systems configured to be located remotely from the plurality of physician consoles;
a first PoP node of the plurality of PoP nodes configured to be connected to: a first physician console of the plurality of physician consoles by a first dedicated connection and a second physician console of the plurality of physician consoles by a second dedicated connection;
a second PoP node of the plurality of PoP nodes configured to be connected to: a first patient-side robotic procedure system of the plurality of patient-side robotic procedure systems by a third dedicated connection; and
a second patient-side robotic procedure system of the plurality of patient-side robotic procedure systems configured to be connected to the first PoP node by a fourth dedicated connection,
the second PoP node configured to be connected to the first PoP node by a fifth connection, and
the first and second POP nodes being further configured to:
enable a first session connection between the first physician console and the first patient-side robotic procedure system to facilitate performing a first robotic assisted medical procedure, the first session connection spanning the first dedicated connection connecting the first physician console to the first PoP node, the third dedicated connection connecting the first patient-side robotic procedure system to the second POP node, and the fifth connection connecting the first and second PoP nodes;
via the first session connection, facilitate transmission of first control data from the first physician console to the first patient-side robotic procedure system, the first control data being used by the first patient-side robotic procedure system to control at least one instrument or at least one imaging system of the first patient-side robotic procedure system;
via the first session connection, facilitate transmission of first image data obtained by the at least one imaging system of the first patient-side robotic procedure system to the first physician console;
enable a second session connection between the second physician console and the second patient-side robotic procedure system to facilitate performing a second robotic assisted medical procedure, the second session connection spanning the second dedicated connection connecting the second physician console to the first PoP node and the fourth dedicated connection connecting the second patient-side robotic procedure system to the first PoP node;
via the second session connection, facilitate transmission of second control data from the second physician console to the second patient-side robotic procedure system, the second control data being used by the second patient-side robotic procedure system to control at least one instrument or at least one imaging system of the second patient-side robotic procedure system; and
via the second session connection, facilitate transmission of second image data obtained by the at least one imaging system of the second patient-side robotic procedure system to the second physician console.

16. The system of claim 15, wherein the first and second session connections facilitate simultaneous transmission of first data over the first session connection and second data over the second session connection such that the first and second robotic assisted medical procedures can be performed simultaneously, and wherein the first data is separated from the second data.

17. The system of claim 15, wherein the at least one of first or second dedicated connections further comprises a direct connection.

18. The system of claim 15, wherein the at least one of first or second dedicated connections comprises a leased line.

19. The system of claim 15, wherein the third connection comprises a dedicated connection.

20. The system of claim 15, further comprising a non-transitory computer readable medium storing instructions that, when executed by at least one processor, cause the at least one processor to:
receive a first request to establish the first session connection between the first physician console and the first patient-side robotic procedure system;
responsive to receiving the first request, establish the first session connection between the first physician console and the first patient-side robotic procedure system;
receive a second request to establish the second session connection between the second physician console and the second patient-side robotic procedure system; and
responsive to receiving the second request, establish the second session connection between the second physician console and second first patient-side robotic procedure system.
